(19) United States
(12) Patent Application Publication
(10) Pub. No.: US 2014/0363460 A1
(43) Pub. Date: Dec. 11, 2014

(54) COMBINED ANTIGEN AND DNA VACCINE FOR PREVENTING AND TREATING RSV INFECTION

(71) Applicants: Bin Wang, Beijing (CN); Xuan Chen, Beijing (CN); Qingling Yu, Beijing (CN)

(72) Inventors: Bin Wang, Beijing (CN); Xuan Chen, Beijing (CN); Qingling Yu, Beijing (CN)

(21) Appl. No.: 14/376,491

(22) PCT Filed: Aug. 6, 2012
(86) PCT No.: PCT/CN2012/001046

§ 371 Date: Aug. 4, 2014

(30) Foreign Application Priority Data
Feb. 10, 2012 (CN) 201210030325.5

(51) Int. Cl. A61K 39/155; C12N 7/00

(52) U.S. Cl. CPC A61K 39/155; C12N 7/00; C12N 2760/18571; C12N 2760/18534
USPC 424/186.1

(57) ABSTRACT

The present disclosure relates to treating and preventing symptoms of respiratory syncytial virus (RSV) infection using a combination vaccine containing an RSV antigen and a DNA encoding the RSV antigen.

### SEQUENCE LISTING

The instant application contains a Sequence Listing which has been submitted in ASCII format and is hereby incorporated by reference in its entirety. Said ASCII copy, created on Aug. 2, 2012, is named “030276-9016_SequenceListing_ST25.txt” and is 116,697 bytes in size.

### FIELD OF THE INVENTION

The present invention relates to treating and preventing respiratory syncytial virus (RSV) infection using a vaccine containing an RSV antigenic peptide and DNA encoding the RSV antigenic peptide.

### BACKGROUND

Human RSV is a Pneumovirus of the family Paramyxoviridae, and a major cause of lower respiratory tract infections amongst children and the elderly, but most commonly in infants less than three years of -age. Conservative estimates suggest approximately 3.3 million cases of respiratory tract disease annually in the elderly in the USA today. Like influenza A disease, RSV epidemics occur every winter, and re-infection with RSV is very common, recurring throughout life. Although the development of a vaccine against RSV is a high priority, no safe and effective vaccine against RSV is available due to the associated vaccine-induced disease (VID). VID is caused by the robust pathogenic inflammation reaction in subjects due to an enhanced inflammatory CD4⁺ T cell responsiveness from RSV antigens.

A safer and effective RSV vaccine should be harmless and induce the right immune responses against the virus. However, the first candidate vaccine, a formalin-inactivated RSV (FI-RSV) vaccine developed in the 1960s induced severe disease following subsequent natural exposure to the virus rather than serving as a vaccine against infection. It resulted in the hospitalization of 80% of the vaccinated infants and two deaths. Peripheral blood lymphocytes from these children showed T lymphocyte hyper-responses compared to naïve or RSV infected controls.

A similar pathogenesis of VID was revealed in animal models that were FI-RSV immunized and then challenged with live RSV. The enhanced histopathologic changes of VID in mice could be abrogated by depletion of CD4⁺ T cells or IL-4 before RSV challenge, further suggesting that the FI-RSV-induced pathologic changes were T cell mediated in this animal model.

F and G proteins of RSV serve as significant neutralizing and major protective antigens, but also result in VID. In particular, it has been found that mice immunized with respiratory syncytial virus (RSV) G glycoprotein (G) exhibit VID following RSV challenge and experienced atypical pulmonary eosinophilia. The deletion of CD4⁺ T cells resulted in less severe disease, suggesting that sequences within the G antigen might contain epitopes that over-stimulate T cell responses.

Although a RSV vaccine is needed where VID occurs upon RSV challenge, a RSV vaccine has not been generated that avoids over-reactive responses such as VID, which further leads to lung pathology damage. An ideal vaccine against RSV infection should meet two requirements: 1) to induce RSV specific neutralizing antibodies; and 2) to not stimulate the excessive T cell responses that cause VID. Many approaches have been tested, such as the use of DNA vaccine, adenoviral vector, Th1 type of adjuvant, and oral delivery, but none have been successful so far. Accordingly, there is a need in the art to develop a vaccine that will induce a neutralizing antibody against RSV infection, but suppress inflammatory CD4⁺ T cells.

### SUMMARY OF THE INVENTION

The present invention is directed to a vaccine against respiratory syncytial virus (RSV) infection comprising an RSV antigenic peptide and a nucleic acid encoding the RSV antigenic peptide wherein the vaccine stimulates iTreg cells (CD4⁺, CD25⁻, FoxP3⁺, IL-10⁺). The RSV antigenic peptide of the vaccine can be F glycoprotein, G glycoprotein, SEQ ID NO: 4 (optimized amino acid RSV G amino acid sequence), SEQ ID NO: 25 (optimized amino acid RSV F amino acid sequence) and functional fragments thereof. The vaccine can have a nucleic acid and antigenic peptide mass ratio from 5:1 and 1:5; and 1:1 and 2:1. The vaccine of the invention can be a nucleic acid. The nucleic acid can be an RNA, DNA or cDNA. The nucleic acid encodes for the RSV antigenic peptide selected from the group consisting of F glycoprotein, G glycoprotein, SEQ ID NO: 4 (optimized amino acid RSV G amino acid sequence), SEQ ID NO: 25 (optimized amino acid RSV F amino acid sequence) and functional fragments thereof. The DNA can be present in a linear expression cassette of a circular plasmid. The plasmid can be selected from the group consisting of pVAX, pcDNA3.0, and proVAX. The plasmid can further comprise a promoter selected from the group consisting of CMV, SV40 early promoter, SV40 later promoter, metallothionein promoter, murine mammary tumor virus promoter, Rous sarcoma virus promoter, and polyhedrin promoter.

The present invention can further be directed to a vaccination kit comprising a vaccine administration device and the vaccine as described above. The vaccine administration device of the kit can be a vaccine gun, or an electroporation device. The kit can comprise minimally-invasive electroporation device.

The present invention can also be directed to a method for preventing or treating respiratory syncytial virus infection in a patient, the method comprising administering to a subject in need thereof the vaccine as described above. The method can further protect the subject from airway hyper-responsiveness (AHR) after respiratory syncytial virus challenge.

The present invention is further directed to a method of inducing neutralizing antibody against respiratory syncytial virus infection and suppressing inflammatory T cells, the method comprising administering the vaccine to a subject in need thereof wherein iTreg cells are induced and auto-reactive CD4+ and CD8+ T cells are suppressed. The vaccine can be administered by electroporation using, for example, a minimally-invasive electroporation device, wherein the electroporation route is selected from the group consisting of intradermal and intramuscular.

The present invention is further directed to a method of treating or preventing vaccine-induced disease in a subject immunized against respiratory syncytial virus (RSV), the method comprising administering the vaccine as described above to a subject in need thereof wherein the subject has been immunized with formalin-inactivated RSV (FI-RSV) or RSV antigen prior to encounter with natural RSV infection. The vaccine can be administered by electroporation using, for example, a minimally-invasive electroporation device, wherein the electroporation route can be intradermal or intramuscular.

### BRIEF DESCRIPTION OF THE DRAWINGS

FIG. 1 shows the eukaryotic expression of the plasmids proVAX/G in NIH/3T3 cells. Total RNA was extracted from NIH/3T3 cells 48 h after transfection with proVAX/G. The templates used for RT-PCR were as follows: lane 1, cDNA from the transfected NIH/3T3 cells with RTs; lane 2, proVAX vector control transfected cells; lane 3, RNA from non-transfected NIH/3T3 cells as a negative control.

FIGS. 2A-2B show the Coomassie blue stains of SDS-PAGE of recombinant protein expression in E. coli BL21(DE3). FIGS. 2A-2B show in lane M, protein molecular markers; lane 1, E. coli BL21(DE3) not induced; lane 2, E. coli BL21(DE3) induced by IPTG under 0.5 mM; lane 3 E. coli BL21(DE3) pET28a(+) not induced; lane 4, E. coli BL21(DE3) pET28a induced by IPTG under 0.5 mM, lane 5, E. coli BL21(DE3) pET28a(+)/G not induced; lanes 6&10, E. coli BL21(DE3) pET28a(+)/G induced by IPTG under 0.5 mM; lane 7, purified polyhistidine-tagged recombinant protein; lane 8, supernatants of E. coli BL21(DE3) lysate transformed by pET28a(+)/G with 0.5 mM IPTG induction; and lane 9, sediment of E. coli BL21(DE3) lysate transformed by pET28a(+)/G with 0.5 mM IPTG induction.

FIG. 3 shows the western blot analysis of polyhistidine-tagged recombinant G protein. E. coli BL21(DE3) lysates obtained from cells grown in LB broth and induced with 0.5 mM IPTG. Protein samples were separated on 12% SDS-PAGE and transferred to a nitrocellulose membrane. The membrane was reacted with goat anti-RSV antibodies and visualized by reacting conjugated secondary anti-goat antibodies. Lane 1, Supernatants of E. coli BL21(DE3) lysate transformed by pET28a(+)/G with 0.5 mM IPTG induced; lane 2, insoluble fraction of E. coli BL21(DE3) lysate transformed by pET28a(+)/G; lane 3, nickel column purified recombinant RSV G protein.

FIGS. 4A-4B show analysis of the humoral response. Serum samples were collected from Balb/c mice on day 7 after the last immunization. RSV G specific antibody (FIG. 4A) and RSV F specific antibody (FIG. 4B) were determined by ELISA using UV-inactivated RSV. Results are presented as an average of data from three groups in triplicate, error bars represent standard deviation. The data shown summarizes one of three experiments, all of which demonstrated similar results. The single asterisk indicates p<0.05, double asterisk indicates p<0.01.

FIG. 5 shows that T cell response impairment is induced by co-immunization of DNA and protein vaccines. T cells were isolated from mice that had been immunized with PSB or proVAX/G or His-G or co-immunized with proVAX+His-G (antigen-mismatched) or proVAX/G+OVA (antigen-mismatched) or co-immunized with proVAX/G+His-G (antigen-matched). The T cells were re-stimulated in vitro using UV-irradiated RSV antigen as specific antigen (third data column from the left), BSA as a non-specific antigen (second data column from the left), or PMA+Ion as a positive control stimulant (first data column from the left). T cell proliferation was determined as described in Example 1. Data shown are representative from three independent experiments. The single asterisk indicates P<0.05, double asterisk indicates p<0.001.

FIGS. 6A-6B show lung RSV titers of immunized mice.

FIGS. 7A-7D show the amount of eosinophils, lymphocytes and monocytes present after RSV challenge compared with total cells. Mice were challenged intranasally with 10⁶ TCID₅₀ live RSV on day 14 after the last immunization. Mice were sacrificed 5 days after RSV challenge and BALs were analyzed. Mean±SEM of total eosinophils (FIG. 7A), monocytes (FIG. 7B), lymphocytes (FIG. 7C) and total cells (FIG. 7D) are shown (n=6 per group). The single asterisk indicates p<0.05 in comparison with the PBS groups.

FIGS. 8A-8D show the amount of eosinophils, lymphocytes and monocytes present after RSV challenge compared with total cells. Mice were challenged intranasally with 10⁶ TCID₅₀ live RSV on day 14 after the last immunization. Mice were sacrificed 5 days after RSV challenge and BALs were analyzed. Mean±SEM of total eosinophils (FIG. 8A), monocytes (FIG. 8B), lymphocytes (FIG. 8C) and total cells (FIG. 8D) are shown (n=6 per group). The single asterisk indicates p<0.05 in comparison with the PBS groups.

FIGS. 9A-9D show whole-body plethysmograpy in response to the intra jugular administration of acetylcholine chloride. 5 days after RSV challenge, airway obstruction was measured by whole-body plethysmograpy in response to the intra jugularadministration of acetylcholine chloride at various doses (at the x-axis) and expressed as dynamic resistance (Rrs) (FIGS. 9A and 9C) and dynamic compliance (Cldyn) (FIGS. 9B and 9D) on the y-axis. The naïve mice were without pre-treatment and challenge. The single asterisk indicates p<0.05 in comparison with other groups.

FIGS. 10A-10H show the histological examination of lung tissues after hematoxylin and eosin staining. 5 days after RSV challenge, mice were euthanized, and the lung tissues were removed and fixed in formalin. Thin sections of paraffin-embedded tissue were cut and stained with hematoxylin and eosin. A representative section (of 6 per group) is shown at each magnification (magnification, 100× or 400×) for tissues from naïve/unchallenged mice (FIG. 10A), PBS mice (FIG. 10B), FI-RSV mice (FIG. 10C), proVAX/G mice (FIG. 10D), His-G mice (FIG. 10E), proVA+His-G mice (FIG. 10F), proVAX/G+OVA mice (FIG. 10G) and proVAX/G+His-G mice (FIG. 10H).

FIGS. 11A-11H show the histological examination of lung tissues after hematoxylin and eosin staining. 5 days after RSV challenge, mice were euthanized, and the lung tissues were removed and fixed in formalin. Thin sections of paraffin-embedded tissue were cut and stained with hematoxylin and eosin. A representative section (of 6 per group) is shown at each magnification (magnification, 100× or 400×) for tissues from naïve/unchallenged mice (FIG. 11A), PBS mice (FIG. 11B), FI-RSV mice (FIG. 11C), proVAX/G mice (FIG. 11D), His-G mice (FIG. 11E), proVA+His-G mice (FIG. 11F), proVAX/G+OVA mice (FIG. 11G) and proVAX/G+His-G mice (FIG. 11H).

FIGS. 12A-12E show the histological examination of lung tissues after hematoxylin and eosin staining. 5 days after RSV challenge, mice were euthanized, and the lung tissues were removed and fixed in formalin. Thin sections of paraffin-embedded tissue were cut and stained with hematoxylin and eosin. A representative section (of 6 per group) is shown at each magnification (magnification, 100× or 400×) for tissues from proVAX/F mice (FIG. 12A), His-F mice (FIG. 12B), proVA+His-F mice (FIG. 12C), proVAX/F+OVA mice (FIG. 12D) and proVAX/F+His-F mice (FIG. 12E).

FIG. 13 shows the histopathologic scores of lung tissue described in FIG. 10. Histopathologic scores (HPS) were evaluated in hematoxylin and eosin stained tissue by a pathologist in a blinded fashion, as described in Example 1. The single asterisk indicates p<0.05 in comparison with other groups.

FIGS. 14A-14B show the weight loss in immunized mice following challenge with live RSV. Following RSV challenge, mice were weighed daily, and weights were normalized to the base weight at day 0. The single asterisk indicates p<0.05 in comparison with other groups.

FIGS. 15A-15D show cytokine production in the lung tissues of mice on day 5 after RSV challenge examined by qPCR. Levels of IL-4 (FIG. 15A), IL-5 (FIG. 15B), IL-13 (FIG. 15C) and IFN-γ (FIG. 15D) were measured. The single asterisk indicates p<0.05, double asterisk indicates p<0.01 and triple asterisk indicates p<0.001.

FIGS. 16A-16N show fluorescence-activated cell sorting (FACS) analysis of T cell phenotypes. Co-immunization induced iTreg cells can mediate antigen-specific T cell suppression in vivo and inhibit allo-mixed lymphocyte reaction in vitro. Lymphocytes were obtained from mice 7 days after the last immunization and stained with anti-CD4-FITC and anti-CD25⁻PE Cy5 mAb, and then intracellular stained with anti-IL-10-PE and anti-Foxp3-APC mAbs. FIG. 16A shows SSC-H versus FSC-H plot. FIG. 16B shows CD4 versus CD25 plot. FIGS. 16C-16N show IL-10 x-axis) and Foxp3 (y-axis) co-expression. Both CD4⁺CD25⁻ (R1) (FIGS. 16I-16N) and CD4⁺CD25⁺ (FIGS. 16C-16H) (R2) T cells were gated for the co-expression of IL-10 and Foxp3. Results shown are representative of three experiments. Percentages represent percent of double-positive cells.

FIGS. 17A-17B show the percentage summaries of CD4⁺CD25⁻Foxp3⁺IL-10⁺ (FIG. 17B) or CD4⁺CD25⁺Foxp3⁺IL-10⁺ (FIG. 17A) T cells in total splenocytes. Triple asterisk indicates p<0.001 when compared with other groups.

FIG. 18 shows suppression mediated by adoptively transferred CD4⁺CD25⁻ donor T cells. CD4⁺CD25⁻ or CD4⁺+CD25⁺ subsets of cells were prepared from Balb/c mice that had been co-immunized with proVAX/G+His-G or from naïve mice, and adoptively transferred into naïve Balb/c mice. The recipient mice were then immunized with His-G 24 hr post-transfer and used to isolate splenocytes 7 days after the immunization. Splenocytes of recipients were restimulated in vitro using UV-irradiated RSV antigen as a specific antigen and the proliferative responses measured by the MTT method.

FIG. 19 shows suppression mediated by adoptively transferred CD4⁺CD25⁻ donor T cells. CD4⁺CD25⁻ or CD4⁺+CD25⁺ subsets of cells were prepared from Balb/c mice that had been co-immunized with proVAX/G+His-G or from naïve mice, and adoptively transferred into naïve Balb/c mice. The recipient mice were then immunized with His-G 24 hr post-transfer and used to isolate splenocytes 7 days after the immunization. Splenocytes of recipient animals (as responder cells) were mixed with mitomycin C-treated splenocytes from naive C57BL/6 mice (as allogeneic stimulator cells) for MLR. The single asterisk indicates p<0.05, double asterisk indicates p<0.01.

FIGS. 20A-20F show the amelioration of pulmonary inflammatory response by adoptive transfer CD4⁺CD25⁻ iTreg cells from the co-immunized mice. CD4⁺CD25⁺ and CD4⁺CD25⁻ T cells were purified from mice co-immunized with proVAX/G+His-G (antigen-matched) or proVAX/G+OVA (antigen-mismatched) on day 7 after the last immunization and these were adoptively transferred intravenously into mice that were previously immunized with His-G, then followed with RSV challenge. 5 days after RSV challenge, the mice were euthanized, and the lung tissues were removed and fixed in formalin. Thin sections of paraffin-embedded tissue were cut and stained with hematoxylin and eosin. A representative section (of 6 per group) is shown at each magnification (magnification, 100× or 400×) for tissues from naïve mice (FIG. 20A), His-G mice (FIG. 20B), “CD4⁺CD25⁺ from proVAX/G+His-G” mice (FIG. 20C), “CD4⁺CD25⁻ from proVAX/G+His-G” mice (FIG. 20D), “CD4⁺CD25⁺ from proVAX/G+OVA” mice (FIG. 20E), and “CD4⁺CD25⁻ from proVAX/G+OVA” mice (FIG. 20F).

FIG. 21 shows the histopathologic scores of the lung tissue described in FIG. 20. Histopathologic scores (HPS) were evaluated in hematoxylin and eosin stained tissue by a pathologist in a blinded fashion, as described in Example 1. The single asterisk indicates p<0.05.

### DETAILED DESCRIPTION

The present invention relates to vaccines for treating and protecting subjects from RSV infection while suppressing vaccine-induced disease due to previous RSV-antigen immunization.

Vaccine induced disease (VID) is due to the predisposition of naive individuals to exacerbate inflammatory responses, including massive lymphocytes infiltrations, pulmonary eosinophilia and type 2 cytokine productions, when they are immunized either with RSV antigen (such as F/G proteins) prior to encounter with the natural RSV infection. However, antibodies have a major role in protection. Passive transfer of neutralizing antibodies can protect immune-deficient individuals or children against RSV infection and the mouse model showed that treatment with anti-RSV neutralizing monoclonal antibody markedly decreased RSV replication and was associated with significant reduction of inflammatory and clinical markers of disease severity. Because of the high costs of passive immunotherapy in children, a vaccine aiming to induce a high level of neutralizing antibodies against RSV is of great interest.

The vaccine of the present invention comprises RSV protein-encoding nucleic acid and its cognate-recombinant RSV protein to protect against RSV infection and to also ameliorate RSV-induced pulmonary inflammatory disorders. The vaccine provides marked reduction of virus replication in the lungs by inducing neutralizing antibody against RSV infection as high as formulin-inactivated-RSV (FI-RSV) infection, but suppressed inflammatory T cells by inducing iTreg (CD4⁺, CD25⁻, FoxP3⁺, IL10⁺) cells. Antigen specific iTreg cells were induced by co-immunization with DNA and protein together. iTreg stimulated cells generated high levels of IL-10, which suggests stimulation of B cells for the neutralizing antibody production. This strategy induces high levels of neutralizing antibody as well as antigen-specific iTreg cells that suppress inflammatory T cells and reduce pathology. Thus, this strategy can protect against RSV infection effectively with minimal, if any, VID.

The use of co-immunization with protein and nucleic acid expressing cognate antigen to induce iTreg in vivo has several advantages, 1) it is comparatively easy, since it only requires administration of a defined protein antigen with its expressing plasmid; 2) it induces a potent iTreg to suppress T cells in an antigen specific manner; 3) it also induces a high level of antibodies. This approach can surmount the main obstacle to an RSV vaccine development since a vaccine with the dual functions, induction of neutralizing antibody and iTreg cells as disclosed, would likely be effective without the VID side-effect.

### 1. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Airway hyper-responsiveness (AHR)” as used herein refers to an abnormality of the airways that allows them to narrow too easily and/or too much in response to a stimulus capable of inducing airflow limitation. AHR can be a functional alteration of the respiratory system resulting from inflammation in the airways or airway remodeling (e.g., such as by collagen deposition). Airflow limitation refers to narrowing of airways that can be irreversible or reversible. Airflow limitation or airway hyperresponsiveness can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma and infiltrative diseases in and around the airways. Many of these causative factors can be associated with inflammation.

“Consensus” or “Consensus Sequence” or “Optimized sequence” as used herein can mean a synthetic nucleic acid sequence, or corresponding polypeptide sequence, constructed based on analysis of an alignment of multiple subtypes of a particular antigen. The sequence can be used to induce broad immunity against multiple subtypes or sertypes of a particular antigen. Synthetic antigens, such as fusion proteins, can be manipulated to consensus sequences (or consensus antigens).

“Fragment” or “functional fragment” as used herein with respect to nucleic acid sequences means a nucleic acid sequence or a portion thereof, that encodes a polypeptide capable of eliciting an immune response in a mammal that cross reacts with a full length wild type RSV antigen. The fragments can be DNA fragments selected from at least one of the various nucleotide sequences that encode protein fragments set forth herein. The fragment can be a nucleic acid sequence encoding a portion of an RSV antigen wherein the portion encodes epitopes that can elicit an immune response in a mammal that cross reacts with a full length wild type RSV antigen.

“Fragment” or “functional fragment” as used herein with respect to polypeptide sequences means a polypeptide capable of eliciting an immune response in a mammal that cross reacts with a full length wild type RSV antigen. Fragments of consensus proteins may comprise at least 10%, at least 20%, at least 30%, at least 40%, at least 50%, at least 60%, at least 70%, at least 80%, at least 90% or at least 95% of a consensus protein. Fragment can be peptide sequence that is portion of the full length polypeptide antigen of a particular RSV protein such as the G glycoprotein or the F glycoprotein and encodes epitopes that can elicit an immune response in a mammal that cross reacts with a full length wild type RSV polypeptide antigen sequence. Fragments of an RSV protein may comprise at least 10%, at least 20%, at least 30%, at least 40%, at least 50%, at least 60%, at least 70%, at least 80%, at least 90% or at least 95% of a full length RSV protein and are capable of generating an immune response.

“nTreg cells” as used herein are T cells (CD4⁺, CD25⁺, Foxp3⁺) whose major mechanism is peripheral tolerance by inhibiting the proliferative responses of convention CD4 and CD8 T cells and can suppress autoimmune and allergic diseases. The transcription factor Foxp3 is necessary and sufficient for immune suppressive activity of the nTreg cells. nTreg cells proliferate readily in vivo in response to antigenic challenge or homeostatic expansion and exhibit higher rates of homeostatic division than convention CD4 T cells. nTreg cells have a high affinity T cell receptors for self antigens allowing these cells to be efficiently activated by self-antigens to suppress autoreactive T cells.

A “peptide” or “polypeptide” as used herein can mean a linked sequence of amino acids and can be natural, synthetic, or a modification or combination of natural and synthetic.

“Treatment” or “treating,” as used herein can mean protecting of an animal from a disease through means of preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

“Subject” as used herein can mean a mammal that wants to or is in need of being immunized with the RSV vaccine. The can be a human, chimpanzee, dog, cat, horse, cow, mouse, or rat.

“Superantigen” as used herein can mean a class of antigens which cause non-specific activation of T-cells resulting in polyclonal T cell activation and massive cytokine release. Compared to a normal antigen-induced T-cell response where 0.001-0.0001% of the body's T-cells are activated, superantigens are capable of activating up to 20% of the body's T-cells. The large number of activated T-cells generates a massive immune response which is not specific to any particular epitope on the superantigen thus undermining one of the fundamental strengths of the adaptive immune system, that is, its ability to target antigens with high specificity. More importantly, the large number of activated T-cells secrete large amounts of cytokines, the most important of which is Interferon gamma (IFN-γ). This excess amount of IFN-γ in turn activates the macrophages. The activated macrophages, in turn, over-produce proinflammatory cytokines such as IL-1, IL-6 and TNF-α. TNF-α is particularly important as a part of the body's inflammatory response. In normal circumstances it is released locally in low levels and helps the immune system defeat pathogens. However when it is systemically released in the blood and in high levels (due to mass T-cell activation resulting from the superantigen binding), it can cause severe and life-threatening symptoms, including shock and multiple organ failure.

“Substantially identical” as used herein can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

A “variant” as used here can mean a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. VACCINE

The present invention is directed to a vaccine comprising a respiratory syncytial virus (RSV) antigen and a nucleic acid encoding the same RSV antigen. The vaccine acts to coimmunize a subject in need thereof with both the virus RSV antigen and nucleic acid encoding the RSV antigen as described below. The vaccine provides the subject in need thereof an antigen-specific immune response against subsequent RSV challenge. The antigen-specific immune response generates high levels of neutralizing antibody comparable to the levels generated by formulin-inactivated-RSV (FI-RSV) vaccine. The vaccine, however, is different from the FI-RSV based vaccine in that iTreg cells (CD4⁺, CD25⁻, FoxP3⁺, IL10⁺) are induced, which results in a significant reduction of pulmonary abnormality in comparison to subjects immunized with FI-RSV vaccines. iTreg stimulated cells generated high levels of IL-10, which suggests stimulation of B cells for the neutralizing antibody production. The iTreg stimulation further results in less inflammation overall and infiltration of T cells into the lungs, characteristic of vaccine-induced disease upon reintroduction of the subject to RSV infection. The vaccine can further prevent and treat respiratory syncytial virus infection in a patient by protecting the subject from airway hyper-responsiveness (AHR) or airway obstruction after RSV challenge. The vaccine can further ameliorate pulmonary histopathogenesis of RSV infection. The vaccine can further reduce the severity of illness after live RSV challenge including combating weight loss.

FI-RSV induces VID due to activation of Th2 (CD4+) type response, which results in the release of B-cell activator and other related cytokines such as IL-3, IL-4, IL-5, CD40 ligands, IL-10, IL-13 granulocyte-macrophage colony-stimulating factor (GM-CSF), and eotaxin. RSV G glycoprotein (G) vaccines induce VID following RSV challenge as both Th1 and Th2 type response are induced. Th1 release macrophage activating effector and other related cytokines such as interferon-gamma (IFN-γ), GM-CSF, tumor necrosis factor alpha (TNF-α) IL-3, TNF-β and IL-2. The vaccine suppresses both Th1 and Th2 responses in an antigen dependent manner yet generate neutralizing antibody titers similar to those generated by Th2 helper cells thereby inducing neutralization and reducing the level of RSV infection.

The vaccine eliminates T cell (Th1, Th2, Th17) responses from the vaccination. The vaccine co-immunizing activity induces iTreg (CD4⁺, CD25⁻, Foxp3⁺) cells. iTreg cells play a role in both regulating the adaptive and innate immune responses to acute infection and in resolving inflammation following viral clearance. iTreg stimulated cells can generate high levels of IL-10, which stimulates B cells for neutralizing antibody production. The vaccine does not induce nTreg cells. RSV infection can actually lead to depletion of CD4+CD25+ nTregs in RSV infected subjects resulting in enhanced disease severity including increased weight loss, recruitment of innate cells to the bronchoalveolar lavage (BAL) fluid and lung, and increased levels of CD4+ and CD8+ T cells producing IFN-γ.

Co-immunization with the vaccine not only protected a subject from RSV challenge, but also suppressed the exacerbated pulmonary inflammation that leads to VID. The prevention of RSV infection and the reduced VID can be due to induction of both high levels of antigen specific neutralizing antibody and of iTreg cells that could suppress T-cell recalled proliferation in an antigen-specific manner. Such co-immunization up-regulated the level of the anti-inflammatory cytokine IL-10, while down regulating the RSV-induced inflammatory cytokines, such as IL-4, IL-5, IL-13 and IFN-γ.

The vaccine can have an RSV encoding nucleic acid to RSV antigen at a mass ratio of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. The vaccine can have an RSV encoding nucleic acid to RSV antigen mass ratio range of 10:1 to 1:10, 9:1 to 1:9, 8:1 to 8:1, 7:1 to 1:7, 6:1 to 1:6, 5:1 to 1:5, 4:1 to 1:4, 3:1 to 1:3, or 2:1 to 1:2.

a. Antigen

The vaccine can comprise an RSV antigen. The RSV antigen can be encoded by a nucleic acid. The nucleic acid can be a DNA, RNA or cDNA that encodes an RSV antigen or fragment thereof. The RSV antigen can be a peptide or protein that causes an immune response. The antigen can trigger the production of an antibody by the immune system. The nucleic acid may encode an RSV antigen that is a fragment of the full length RSV antigen, and has epitopes present on the RSV antigen capable of triggering an immune response. The RSV antigen may be fragment of the full length RSV antigen, and has epitopes present on the RSV antigen capable of triggering an immune response. The antibody generated by the immune response can then kill or neutralize the antigen that is recognized as a foreign and potentially harmful invader. The RSV antigen can be any molecule or molecular fragment that can be bound by a major histocompatibility complex (MHC) and presented to a T-cell receptor. The RSV antigen can be an immunogen, which is a molecule that is able to provoke an adaptive immune response.

According to the invention, the RSV antigen can be derived from any subtype, such as subtype A or subtype B, or any strain of naturally occurring or recombinant RSV, preferably from human RSV strains. Examples of RSV strains include, but not limited to, strains of subtype A, such as Long (ATCC® VR-26), A2 (ATCC® VR-1540), RSB1734, RSB5857, RSB6190, RSB6256, RSB642, and RSB6614, strains of subtype B, such as B1, 18537, 8/60, and 9320, S2, RSS-2, RSP112/Sweden/02-03, RSP120/Sweden/02-03, RSP121/Sweden/02-03, RSP122/Sweden/02-03, RSP13/Sweden/02-03), RSP140/Sweden/02-03, RSP16/Sweden/02-03, RSP171/Sweden/02-03, RSP183/Sweden/02-03, RSP191/Sweden/02-03, RSP199/Sweden/02-03, RSP212/Sweden/02-03, RSP41/Sweden/02-03, RSP45/Sweden/02-03, RSP56/Sweden/02-03, RSP58/Sweden/02-03, RSP67/Sweden/02-03 and RSP94/Sweden/02-03.

This disclosure demonstrates the protective efficacy obtained by co-immunization with DNA vaccine encoding RSV F antigen together with its F protein or by co-immunization with DNA vaccine encoding RSV G antigen together with its G protein, which gave protection against RSV challenge that was comparable to that obtained with FI-RSV. Other RSV antigens can have similar efficacy. The protection can be ascribed to induction of high levels of neutralizing antibody, similar to FI-RSV, however, the co-immunization protection is associated with the induction of iTreg and resulted in less inflammation and infiltration of T cells into lungs, i.e., significant reduction of pulmonary abnormality.

The vaccine includes an RSV antigenic peptide and DNA encoding the RSV antigenic peptide. In some embodiments, the RSV antigenic peptide is selected from the group consisting of human RSV F and G proteins.

Also provided herein is a DNA that encodes the antigen. The DNA can include an encoding sequence that encodes the antigen. The DNA can also include additional sequences that encode linker or tag sequences that are linked to the antigen by a peptide bond.

(1) RSV F and G Antigens

The RSV antigen can be a human RSV fusion protein (also referred to herein as “RSV F”, “RSV F protein” and “F protein”), or fragment or variant thereof. The human RSV fusion protein is conserved between RSV subtypes A and B. The RSV antigen can be a RSV F protein, or fragment or variant thereof, from the RSV Long strain (GenBank AAX23994.1; SEQ ID NO: 1) encoded by the nucleotide sequence of SEQ ID NO: 26, which corresponds to position 5660-7384 of GenBank AY911262.1 (SEQ ID NO: 6). The RSV antigen can be a RSV F protein from the RSV A2 strain (GenBank AAB59858.1), or a fragment or variant thereof. The RSV antigen can be a monomer, a dimer or trimer of the RSV F protein, or a fragment or variant thereof. The RSV antigen can be an optimized amino acid RSV F amino acid sequence, or fragment or variant thereof. For example, the RSV antigen can be amino acid 412-524 of RSV F or an optimized amino acid sequence thereof. The RSV antigen can be a fusion protein of a dimer of amino acid 412-524 of RSV F or an optimized amino acid sequence thereof, such as SEQ ID NO: 25. The RSV antigen can be an RSV F protein encoded by SEQ ID NO: 23, 24 or 26.

The postfusion form of RSV F elicits high titer neutralizing antibodies in immunized animals and protects the animals from RSV challenge. The present invention utilizes this immunoresponse in the claimed vaccines. According to the invention, the RSV F protein can be in a prefusion form or a postfusion form.

The RSV antigen can be human RSV attachment glycoprotein (also referred to herein as “RSV G”, “RSV G protein” and “G protein”), or fragment or variant thereof. The human RSV G protein differs between RSV subtypes A and B. The antigen can be RSV G protein, or fragment or variant thereof, from the RSV Long strain (GenBank AAX23993; SEQ ID NO: 2), encoded by the nucleotide sequence of SEQ ID NO: 19, which corresponds to position 4687-5583 of GenBank AY911262.1 (SEQ ID NO: 6). The RSV antigen can be RSV G protein from: the RSV subtype B isolate H5601 (SEQ ID NO: 46), the RSV subtype B isolate H1068 (SEQ ID NO: 48), the RSV subtype B isolate H5598 (SEQ ID NO: 50), the RSV subtype B isolate H1123 (SEQ ID NO: 52), or a fragment or variant thereof. The RSV antigen can be an optimized amino acid RSV G amino acid sequence, or fragment or variant thereof. For example, the antigen can be amino acid 67-298 of RSV G protein or an optimized amino acid sequence thereof, such as SEQ ID NO: 4. The RSV antigen can be an RSV G protein encoded by SEQ ID NOs: 3, 5, 19, 20, 21, 22, 45, 47, 49, or 51.

(2) Other RSV Antigens

The RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1; SEQ ID NO: 28) encoded by the nucleotide sequence of SEQ ID NO: 27, which corresponds to position 99-518 of GenBank AY911262.1 (SEQ ID NO: 6).

The RSV antigen can be human RSV non-structural protein 2 (“NS2 protein”), or fragment or variant thereof. For example, the RSV antigen can be RSV NS2 protein, or fragment or variant thereof, from the RSV Long strain (GenBank AAX23988.1; SEQ ID NO: 30) encoded by the nucleotide sequence of SEQ ID NO: 29, which corresponds to position 628-1002 of GenBank AY911262.1 (SEQ ID NO: 6).

The RSV antigen can be human RSV nucleocapsid (“N”) protein, or fragment or variant thereof. For example, the RSV antigen can be RSV N protein, or fragment or variant thereof, from the RSV Long strain (GenBank AAX23989.1; SEQ ID NO: 32) encoded by the nucleotide sequence of SEQ ID NO: 31, which corresponds to position 1140-2315 of GenBank AY911262.1 (SEQ ID NO: 6).

The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1; SEQ ID NO: 34) encoded by the nucleotide sequence of SEQ ID NO: 33, which corresponds to position 2346-3071 of GenBank AY911262.1 (SEQ ID NO: 6).

The RSV antigen can be human RSV Matrix protein (“M”) protein, or fragment or variant thereof. For example, the RSV antigen can be RSV M protein, or fragment or variant thereof, from the RSV Long strain (GenBank AAX23991.1; SEQ ID NO: 36) encoded by the nucleotide sequence of SEQ ID NO: 35, which corresponds to position 3261-4031 of GenBank AY911262.1 (SEQ ID NO: 6).

The RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1; SEQ ID NO: 38) encoded by the nucleotide sequence of SEQ ID NO: 37, which corresponds to position 4302-4496 of GenBank AY911262.1 (SEQ ID NO: 6).

The RSV antigen can be human RSV Matrix protein2-1 (“M2-1”) protein, or fragment or variant thereof. For example, the RSV antigen can be RSV M2-1 protein, or fragment or variant thereof, from the RSV Long strain (GenBank AAX23995.1; SEQ ID NO: 40) encoded by the nucleotide sequence of SEQ ID NO: 39, which corresponds to position 7605-8189 of GenBank AY911262.1 (SEQ ID NO: 6).

The RSV antigen can be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1; SEQ ID NO: 42) encoded by the nucleotide sequence of SEQ ID NO: 41, which corresponds to position 8158-8430 of GenBank AY911262.1 (SEQ ID NO: 6).

The RSV antigen can be human RSV Polymerase L (“L”) protein, or fragment or variant thereof. For example, the RSV antigen can be RSV L protein, or fragment or variant thereof, from the RSV Long strain (GenBank AAX23996.1; SEQ ID NO: 44) encoded by the nucleotide sequence of SEQ ID NO: 43, which corresponds to position 8497-14994 of GenBank AY911262.1 (SEQ ID NO: 6).

The RSV antigen can be an optimized amino acid sequence of NS1, NS2, N, P, M, SH, M2-1, M2-2, or L protein.

(3) Summarization of RSV Antigens

The RSV antigen can be a human RSV protein or recombinant antigen, such as any one of the proteins encoded by the human RSV genome (SEQ ID NO: 6). The antigen can be a human RSV or recombinant protein or consensus thereof, a fragment thereof, or a variant thereof, of SEQ ID NOs: 1, 2, 4, 25, 28, 30, 32, 34, 36, 38, 40, 42, 44, 46, 48, 50, or 52. The RSV antigen can be a human RSV or recombinant protein or consensus thereof, a fragment thereof, or a variant thereof, encoded by SEQ ID NO: 3, 5, 19, 20, 21, 22, 23, 24, 26, 27, 29, 31, 33, 35, 37, 39, 41, 43, 45, 47, 49, or 51.

SEQ ID NOs: 1 and 26 represent the amino acid and nucleotide sequences for the RSV F protein from the RSV Long strain.

SEQ ID NOs: 2 and 19 represent the amino acid and nucleotide sequences for the RSV G protein from the RSV Long strain.

SEQ ID NOs: 3, 5, 20, 21, and 22 represent nucleotide sequences encoding the optimized amino acid RSV G amino acid sequence of SEQ ID NO: 4. SEQ ID NO: 20 encodes the amino acid sequence of SEQ ID NO: 4. SEQ ID NOs: 3 and 21 represent prokaryotic (E. coli) codon-optimized sequences encoding the amino acid sequence of SEQ ID NO: 4 and SEQ ID NOs: 5 and 22 represent eukaryotic (mouse) codon-optimized sequences encoding the amino acid sequence of SEQ ID NO: 4.

SEQ ID NO: 6 represents the nucleotide sequence of the human RSV genome of the RSV Long strain.

SEQ ID NOs: 23 and 24 represent nucleotide sequences encoding the optimized amino acid RSV F amino acid sequence of SEQ ID NO: 25. SEQ ID NO: 23 encodes the amino acid sequence of SEQ ID NO: 25. SEQ ID NO: 24 represents a prokaryotic (E. coli) codon-optimized sequence encoding the amino acid sequence of SEQ ID NO: 25.

SEQ ID NOs: 27 and 28 represent the amino acid and nucleotide sequences for the RSV NS1 protein from the RSV Long strain.

SEQ ID NOs: 29 and 30 represent the amino acid and nucleotide sequences for the RSV NS2 protein from the RSV Long strain.

SEQ ID NOs: 31 and 32 represent the amino acid and nucleotide sequences for the RSV N protein from the RSV Long strain.

SEQ ID NOs: 33 and 34 represent the amino acid and nucleotide sequences for the RSV P protein from the RSV Long strain.

SEQ ID NOs: 35 and 36 represent the amino acid and nucleotide sequences for the RSV M protein from the RSV Long strain.

SEQ ID NOs: 37 and 38 represent the amino acid and nucleotide sequences for the RSV SH protein from the RSV Long strain.

SEQ ID NOs: 39 and 40 represent the amino acid and nucleotide sequences for the RSV M2-1 protein from the RSV Long strain.

SEQ ID NOs: 41 and 42 represent the amino acid and nucleotide sequences for the RSV M2-2 protein from the RSV Long strain.

SEQ ID NOs: 43 and 44 represent the amino acid and nucleotide sequences for the RSV L protein from the RSV Long strain.

SEQ ID NOs: 45 and 46 represent the amino acid and nucleotide sequences for the RSV G protein from the RSV subtype B isolate H5601.

SEQ ID NOs: 47 and 48 represent the amino acid and nucleotide sequences for the RSV G protein from the RSV subtype B isolate H1068.

SEQ ID NOs: 49 and 50 represent the amino acid and nucleotide sequences for the RSV G protein from the RSV subtype B isolate H5598.

SEQ ID NOs: 51 and 52 represent the amino acid and nucleotide sequences for the RSV G protein from the RSV subtype B isolate H1123.

b. Vectors

The vaccine can comprise the RSV nucleic acid encoding the antigen and this nucleic acid can be located in a vector. The vector therefore can include the nucleic acid encoding the antigen described above. The vector can be capable of expressing the antigen. The vector can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

A particular DNA vector comprising the DNA encoding the antigen is proVAX/G (SEQ ID NO: 22), which corresponds to the coding region for 67-298 amino acid of RSV G glycoprotein (full length nucleotide sequence corresponds to position 4687-5583 of GenBank: AY911262.1 (RSV genome; SEQ ID NO: 6; full length nucleotide sequence (SEQ ID NO: 19)). Another particular DNA vector comprising the DNA encoding the antigen is proVAX/F (SEQ ID NO: 23), which corresponds to a dimer of the coding region of 412-524 amino acid of RSV F fusion protein (full length nucleotide sequence corresponds to position 5660-7384 of GenBank: AY911262.1 (SEQ ID NO: 6); full length F fusion protein nucleotide sequence (SEQ ID NO: 26)). The coding region can be codon optimized, i.e., codons which are employed more frequently in one organism relative to another organism, a distantly related organism, as well as modifications to add or modify Kozak sequences and/or introns, and/or to remove undesirable sequences, for instance, potential transcription factor binding sites.

The vectors can have expression signals such as a strong promoter, a strong termination codon, adjustment of the distance between the promoter and the cloned gene, and the insertion of a transcription termination sequence and a PTIS (portable translation initiation sequence).

(1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which can be operably linked to termination signals. The vector can also contain sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter which initiates transcription only when the host cell is exposed to some particular external stimulus. In the case of a multicellular organism, the promoter can also be specific to a particular tissue or organ or stage of development.

(2) Circular or Linear Vectors

The vector can be circular plasmid, which can transform a target cell by integration into the cellular genome or exist extrachromosomally (e.g. autonomous replicating plasmid with an origin of replication).

The vector can be pVAX, pcDNA3.0, or proVAX, or any other expression vector capable of expressing the DNA and enabling a cell to translate the sequence to the antigen that is recognized by the immune system. Also provided herein is a linear nucleic acid vaccine, or linear expression cassette (“LEC”), that is capable of being efficiently delivered to a subject via electroporation and expressing one or more desired antigens. The LEC can be any linear DNA devoid of any phosphate backbone. The DNA can encode one or more antigens. The LEC can contain a promoter, an intron, a stop codon, a polyadenylation signal. The expression of the antigen can be controlled by the promoter. The LEC can not contain any antibiotic resistance genes and/or a phosphate backbone. The LEC can not contain other nucleic acid sequences unrelated to the desired antigen gene expression.

The LEC can be derived from any plasmid capable of being linearized. The plasmid can be capable of expressing the antigen. The plasmid can be pNP (Puerto Rico/34) or pM2 (New Calcdonia/99). The plasmid can be pVAX, pcDNA3.0, or proVAX, or any other expression vector capable of expressing the DNA and enabling a cell to translate the sequence to the antigen that is recognized by the immune system.

The LEC can be perM2. The LEC can be perNP. perNP and perMR can be derived from pNP (Puerto Rico/34) and pM2 (New Calcdonia/99), respectively. The LEC can be combined with antigen at a mass ratio of between 5:1 and 1:5, or of between 1:1 to 2:1.

(3) Promoter, Intron, Stop Codon, and Polyadenylation Signal

The vector can have a promoter. A promoter can be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter can be positioned about the same distance from the transcription start in the vector as it is from the transcription start site in its natural setting. However, variation in this distance can be accommodated without loss of promoter function.

The promoter can be operably linked to the nucleic acid sequence encoding the antigen and signals required for efficient polyadenylation of the transcript, ribosome binding sites, and translation termination. The promoter can be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector can include an enhancer and an intron with functional splice donor and acceptor sites. The vector can contain a transcription termination region downstream of the structural gene to provide for efficient termination. The termination region can be obtained from the same gene as the promoter sequence or can be obtained from different genes.

c. Excipients and Other Components of the Vaccine

The vaccine can further comprise other components such as a transfection facilitating agent, a pharmaceutically acceptable excipient, an adjuvant. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent can be a polyanion, polycation, including poly-L-glutamate (LGS), or lipid. The transfection facilitating agent can be poly-L-glutamate. The poly-L-glutamate can be present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA plasmid vaccines can also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are delivered as proteins in combination with the plasmid above in the vaccine. The adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRCS, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine can further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 3. METHOD OF VACCINATION TO TREAT OR PREVENT

The present invention is also directed to methods for preventing and treating respiratory syncytial virus (RSV) infection in a patient. The method includes administering to a subject in need thereof a vaccine against RSV, as described herein. The vaccine includes an RSV antigenic peptide and a nucleic acid encoding the RSV antigenic peptide. The co-immunization vaccine can stimulate iTreg cells, including but not limited to, CD4⁺, CD25⁻, FoxP3⁺, IL-10⁺.

The co-immunization can be used to treat or ameliorate vaccine-induced disease (VID) which is due to the predisposition of naive individuals to exacerbation of inflammatory responses, including massive lymphocytes infiltrations, pulmonary eosinophilia and type 2 cytokine productions, when they are immunized either with FI-RSV or its G antigen or its F antigen prior to encounter with the natural RSV infection. VID amelioration can be indicated by reduced histomorphological changes in lung following virus challenge, and or the association with significant inhibition of the proliferation and infiltration of lymphocytes and eosinophils. For example, amelioration of VID can be caused by the induction of G antigen specific iTreg cells exhibiting a CD4⁺CD25⁻FoxP3⁺IL-10⁺ phenotype. iTreg stimulated cells can generate high levels of IL-10, which stimulates B cells for neutralizing antibody production.

The co-immunization can induce the up-regulation of anti-inflammatory cytokine Il-10 levels, while down regulating the RSV-induced inflammatory cytokines, such as IL-4, Il-5, IL-13 and IFN-γ.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce iTreg responses. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccines can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation can be carried out via a minimally invasive device.

The minimally invasive electroporation device (“MID”) can be an apparatus for injecting the vaccine described above and associated fluid into body tissue. The device can comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection can be reduced due to the distribution of the DNA being injected over a larger area.

The MID can inject the vaccine into tissue without the use of a needle. The MID can inject the vaccine as a small stream or jet with such force that the vaccine pierces the surface of the tissue and enters the underlying tissue and/or muscle. The force behind the small stream or jet can be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. No. 6,520,950; U.S. Pat. No. 7,171,264; U.S. Pat. No. 6,208,893; U.S. Pat. No. 6,009,347; U.S. Pat. No. 6,120,493; U.S. Pat. No. 7,245,963; U.S. Pat. No. 7,328,064; and U.S. Pat. No. 6,763,264, the contents of each of which are herein incorporated by reference.

The MID can comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport can be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector can be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID can have needle electrodes that electroporate the tissue. By pulsing between multiple pairs of electrodes in a multiple electrode array, for example, set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles can be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells can be used. Such a device and system is described in U.S. Pat. No. 6,763,264, the contents of which are herein incorporated by reference. Alternatively, a single needle device can be used that allows injection of the DNA and electroporation with a single needle resembling a normal injection needle and applies pulses of lower voltage than those delivered by presently used devices, thus reducing the electrical sensation experienced by the patient.

The MID can comprise one or more electrode arrays. The arrays can comprise two or more needles of the same diameter or different diameters. The needles can be evenly or unevenly spaced apart. The needles can be between 0.005 inches and 0.03 inches, between 0.01 inches and 0.025 inches; or between 0.015 inches and 0.020 inches. The needle can be 0.0175 inches in diameter. The needles can be 0.5 mm, 1.0 mm, 1.5 mm, 2.0 mm, 2.5 mm, 3.0 mm, 3.5 mm, 4.0 mm, or more spaced apart.

The MID can consist of a pulse generator and a two or more-needle vaccine injectors that deliver the vaccine and electroporation pulses in a single step. The pulse generator can allow for flexible programming of pulse and injection parameters via a flash card operated personal computer, as well as comprehensive recording and storage of electroporation and patient data. The pulse generator can deliver a variety of volt pulses during short periods of time. For example, the pulse generator can deliver three 15 volt pulses of 100 ms in duration. An example of such a MID is the Elgen 1000 system by Inovio Pharmaceuticals, which is described in U.S. Pat. No. 7,328,064, the contents of which are herein incorporated by reference.

The MID can be a CELLECTRA (Inovio Pharmaceuticals) device and system, which is a modular electrode system, that facilitates the introduction of a macromolecule, such as a DNA, into cells of a selected tissue in a body or plant. The modular electrode system can comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The Elgen 1000 system can comprise device that provides a hollow needle; and fluid delivery means, wherein the apparatus is adapted to actuate the fluid delivery means in use so as to concurrently (for example, automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection can be carried out after the needles have been inserted through the skin of the subject to be treated and while they are inserted further into the body tissue.

Suitable tissues into which fluid can be injected by the apparatus of the present invention include tumor tissue, skin or liver tissue but can be muscle tissue.

The apparatus can further comprise a needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus can include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means can comprise an ultrasound probe. The sensing means can comprise a means for sensing a change in impedance or resistance. In this case, the means can not as such record the depth of the needle in the body tissue but will rather be adapted to sense a change in impedance or resistance as the needle moves from a different type of body tissue into muscle. Either of these alternatives provides a relatively accurate and simple to operate means of sensing that injection can commence. The depth of insertion of the needle can further be recorded if desired and could be used to control injection of fluid such that the volume of fluid to be injected is determined as the depth of needle insertion is being recorded.

The apparatus can further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means comprise piston driving means adapted to inject fluid at a controlled rate. The piston driving means could for example be activated by a servo motor. The piston driving means can be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it can further comprise a means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied can be reduced while achieving a good fit between the electric field and the fluid.

### 4. METHOD FOR VACCINATING AGAINST RSV INFECTION

The present invention is also directed to methods for vaccinating a subject against RSV infection. The method includes administering to a subject in need thereof a vaccine against RSV, as described herein. The vaccine can include an RSV antigenic peptide and a nucleic acid encoding the RSV antigenic peptide. The vaccine can stimulate iTreg cells, including but not limited to CD4⁺, CD25⁻, FoxP3⁺, IL-10⁺, which in tern generate an antigen-specific response. The iTreg stimulated cells can generate high levels of IL-10, which stimulates B cells for neutralizing antibody production

### 5. METHOD FOR INDUCING NEUTRALIZING ANTIBODY AGAINST RSV INFECTION AND SUPPRESSING INFLAMMATORY T CELLS

The present invention is also directed to methods for inducing neutralizing antibody against RSV infection and suppressing inflammatory T cells in a subject. The method includes administering to a subject in need thereof a vaccine against RSV, as described herein. The vaccine can include an RSV antigenic peptide and a nucleic acid encoding the RSV antigenic peptide. The vaccine can stimulate iTreg cells, including but not limited to CD4⁺, CD25⁻, FoxP3⁺, IL-10⁺. iTreg stimulated cells can generate high levels of IL-10, which stimulates B cells for neutralizing antibody production. Suppressing inflammatory T cell includes inducing iTreg cells.

The method takes advantage of the antigen-specific immune response generated by coimmunizing a subject with both an RSV antigen and a nucleic acid encoding the same RSV antigen, as discussed above.

### 6. METHOD FOR SUPPRESSING AUTO-REACTIVE CD4+ T CELL INDUCTION AFTER RSV CHALLENGE

The present invention is also directed to methods for suppressing auto-reactive CD4+ T cell induction after RSV challenge in a subject. The method includes administering to a subject in need thereof a vaccine against RSV, as described herein. The vaccine can include an RSV antigenic peptide and a nucleic acid encoding the RSV antigenic peptide. The vaccine can stimulate iTreg cells, including but not limited to CD4⁺, CD25⁻, FoxP3⁺, IL-10⁺. iTreg stimulated cells can generate high levels of IL-10, which stimulates B cells for neutralizing antibody production. Suppressing inflammatory T cell includes suppressing auto-reactive CD4+ and CD8+ T cells.

### 7. METHOD OF AMELIORATING VACCINE-INDUCED DISEASE (VID)

The present invention is also directed to methods of ameliorating VID. The method includes administering to a subject in need thereof a vaccine against RSV, as described herein. The vaccine can include an RSV antigenic peptide and a nucleic acid encoding the RSV antigenic peptide. The vaccine can stimulate iTreg cells, including but not limited to CD4⁺, CD25⁻, FoxP3⁺, IL-10⁺. iTreg stimulated cells can generate high levels of IL-10, which stimulates B cells for neutralizing antibody production. The subject can be immunized with formalin-inactivated RSV (FI-RSV) or RSV antigen prior to encounter with natural RSV infection.

### 8. METHOD FOR PROTECTING A SUBJECT FROM AIRWAY HYPER-RESPONSIVENESS (AHR) AFTER RSV CHALLENGE

The present invention is also directed to methods for protecting a subject from airway hyper-responsiveness (AHR) after RSV challenge. The method includes administering to a subject in need thereof a vaccine against RSV, as described herein. The vaccine can include an RSV antigenic peptide and a nucleic acid encoding the RSV antigenic peptide. The vaccine can stimulate iTreg cells, including but not limited to CD4⁺, CD25⁻, FoxP3⁺, IL-10⁺. iTreg stimulated cells can generate high levels of IL-10, which stimulates B cells for neutralizing antibody production. The subject can be immunized with formalin-inactivated RSV (FI-RSV) or RSV antigen prior to encounter with natural RSV infection. The method takes advantage of the antigen-specific immune response generated by coimmunizing a subject with both an RSV antigen and a nucleic acid encoding the same RSV antigen, as discussed above.

RSV can trigger AHR in a subject previously sensitized to RSV. Sensitization to RSV refers to being previously exposed one or more times to RSV such that an immune response is developed against RSV. Responses associated with an allergic reaction (e.g., histamine release, rhinitis, edema, vasodilation, bronchial constriction, airway inflammation), typically do not occur when a naive individual is exposed to RSV for the first time, but once a cellular and humoral immune response is produced against RSV, the individual is “sensitized” to RSV. AHR or airway obstruction can occur when the sensitized subject is re-exposed to RSV. Once a subject is sensitized to RSV, the allergic reactions can become worse with each subsequent exposure to RSV, because each re-exposure not only produces allergic symptoms, but further increases the level of antibody produced against RSV and the level of T cell response against RSV.

A subject that is at risk of developing airway hyperresponsiveness is a subject that has been exposed to, or is at risk of being exposed to, RSV that is sufficient to trigger AHR, but does not yet display a measurable or detectable characteristic or symptom of AHR. A mammal that is at risk of developing RSV-induced AHR is a mammal that has been previously sensitized to RSV, and that has been exposed to, or is at risk of being exposed to, an amount of RSV that is sufficient to trigger AHR (i.e., a triggering, or challenge dose of RSV), but does not yet display a measurable or detectable characteristic or symptom of AHR.

Inflammation is typically characterized by the release of inflammatory mediators (e.g., cytokines or chemokines) which recruit cells involved in inflammation to a tissue. AHR after RSV challenge involves the elicitation of one type of immune response (e.g., a Th2-type immune response) against RSV, which can result in the release of inflammatory mediators that recruit cells involved in inflammation in a mammal, the presence of which can lead to tissue damage and sometimes death. A Th2-type immune response is characterized in part by the release of cytokines which include IL-4, IL-5 and IL-13.

### 9. COMBINED THERAPIES

As used herein, the term “in combination” in the context of the administration of other therapies refers to the use of more than one therapy. The use of the term “in combination” does not restrict the order in which therapies are administered to a subject with an infection. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a RSV infection, otitis media or a respiratory condition related thereto. Any additional therapy can be administered in any order with the other additional therapies. In certain embodiments, the vaccines of the invention can be administered in combination with one or more therapies (e.g., therapies that are not the vaccines of the invention that are currently administered to prevent, treat, manage, and/or ameliorate a RSV infection (e.g., acute RSV disease or a RSV URI and/or LRI, otitis media, and/or a symptom or respiratory condition or other symptom related thereto).

Non-limiting examples of therapies that can be administered in combination with a vaccine of the invention include at least one of any suitable and effective amount of a composition or pharmaceutical composition comprising at least one RSV antibody to a cell, tissue, organ, animal or patient in need of such modulation, treatment or therapy, optionally further comprising at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluororquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist, or any other agent listed in the U.S. Pharmacopoeia and/or Physician's Desk Reference. Non-limiting examples of such cytokines include, but are not limited to, any of IL-1 to IL-23. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

### 10. KIT

Provided herein is a kit, which can be used for vaccinating a subject. The kit can comprise a vaccine, which includes an antigenic peptide and a DNA encoding the antigenic peptide, and a MID. The kit can further comprise instructions for using the kit and conducting the analysis, monitoring, or treatment.

The kit can also comprise one or more containers, such as vials or bottles, with each container containing a separate reagent. The kit can further comprise written instructions, which can describe how to perform or interpret an analysis, monitoring, treatment, or method described herein. The kit can further comprise a vaccine administration device. The vaccine administration device can be a vaccine gun, a needle for administering the vaccine, or a electroporation device linked to a conduit for delivering the vaccine.

The present invention has multiple aspects, illustrated by the following non-limiting examples.

### EXAMPLES

### Example 1

### Materials and Methods

The following is a description of the materials and methods used in the below-identified Examples 2-8.

With respect to the animals and cells, female Balb/c mice aged 6-8 weeks were purchased from the Animal Institute of the Chinese Medical Academy (Beijing, China), cared for under a 12-hour light cycle, and fed with pathogen-free food and water. All animal protocols were approved by the Animal Welfare Committee of China Agricultural University. NIH/3T3 cell line (American Type Culture Collection (ATCC), Rockville, Md., USA CCL-1658) and HEp-2 (ATCC, CCL-23) cells were maintained in Dulbecco's modified Eagle's medium (DMEM) (Gibco/BRL, NY, USA) supplemented with 10% fetal calf serum (FCS) and penicillin/streptomycin (Gibco/BRL) in a humidified incubator set at 37° C. and 5% CO₂.

With respect to the bacterial strains and plasmids, E. coli TOP 10 (TIANGEN Biotech LTD, Beijing, China) was used as the host strain for manipulation and E. coli BL21 (DE3) (TIANGEN) was used as the expression strain. Eukaryotic expression vector proVAX was previously constructed in this laboratory with a cytomegalovirus (CMV) promoter and an hCG-β leader sequence (Du et al. The Journal of Gene Medicine 9: 136-146 (2007)). Prokaryotic expression vector pET28a(+) (Novagen Inc, WI, USA) contains the T7 promoter and allows expression of recombinant protein fused to a polyhistidine-tag. All bacterial cultures were carried out in shake-flasks using Luria-Bertani medium (Sangon Biological Engineering Technology & Services Co., Ltd., Shanghai, China) supplemented with kanamycin where appropriate. Protein expression was induced by Isopropyl β-D-1-thiogalactopyranoside (IPTG) purchased from Invitrogen (CA, USA).

With respect to plasmid construction and eukaryotic expression, a gene encoding an optimized version of amino acids 67-298 of RSV G glycoprotein was used (full length RSV G nucleotide sequence corresponds to position 4687-5583 of GenBank: AY911262.1 (RSV genome; SEQ ID NO: 6); RSV G glycoprotein amino acid sequence (GenBank AAX23993; SEQ ID NO: 2); RSV G glycoprotein nucleotide sequence (SEQ ID NO: 19)). The nucleotide sequence (SEQ ID NO: 20) of optimized amino acid RSV G amino acid sequence was mouse-codon optimized (SEQ ID NO: 22) and synthesized by outsourcing (Sangon, Shanghai, China). Codon optimization not only eliminated the rare codons of the G fragment, but also removed the 66-amino acid transmembrane domain of the N-terminus (Ghildyal et al. Journal of General Virology 86: 1879-1884 (2005)). The codon-optimized gene was subcloned into a eukaryotic expression vector, proVAX, at EcoR I and Xho I restriction sites and designated as proVAX/G. Its correctness was confirmed by restriction digestions and sequencing. Purified plasmid was transfected into NIH/3T3 cells with Lipofectamine according to the manufacturer's instructions (Invitrogen, CA, USA). The transfectants were harvested after 48 h and total cellular RNA was extracted according to a previously described protocol (Jin et al. Vaccine 22: 2925-2935 (2004)). The expression of the gene of interest was detected by reverse-transcription polymerase chain reaction (RT-PCR) with specific primer sets: forward primer, 5′-ATGCATAAGGTGACTCCT-3′ (SEQ ID NO: 7), reverse primer, 5′-TTACTGCCGTGGGGTGTT-3′ (SEQ ID NO: 8).

A gene encoding an optimized version of amino acids 412-524 of RSV F fusion protein was used (full length RSV F fusion protein nucleotide sequence corresponds to 5660-7384 of GenBank: AY911262.1 (SEQ ID NO: 6) RSV F fusion protein amino acid sequence (GenBank AAX23994.1; SEQ ID NO: 1); RSV F fusion protein nucleotide sequence (SEQ ID NO: 26)). The nucleotide sequence (SEQ ID NO: 23) of the optimized amino acid RSV F amino acid sequence was codon optimized for prokaryotic (i.e., E. coli) expression (SEQ ID NO: 24).

With respect to plasmid construction and prokaryotic expression, a prokaryotic codon-optimized gene encoding amino acids at 67-298 of RSV G glycoprotein (SEQ ID NO: 21) was subcloned into the prokaryotic expressing vector by a similar procedure and designated as pET28a(+)/G. The prokaryotic codon-optimized gene encoding amino acids at 412-524 of RSV F (SEQ ID NO: 24) was subcloned into the prokaryotic expressing vector by a similar procedure and designated as pET28a(+)/F. Its correctness was confirmed by restriction digestions and sequencing. E. coli BL21 (DE3) competent cells were transformed with pET28a(+)/G by a standard method (Molecular cloning: A laboratory manual, 2nd edn: by J. Sambrook, E. F. Fritsch and T. Maniatis, Cold Spring Harbor Laboratory Press, 1989) and protein expression was induced with 0.5 mmol/L IPTG at 37° C. After 4 h, cells were collected, washed and resuspended in PBS. The soluble fraction and the insoluble fraction containing inclusion bodies were isolated from the cells after sonication. Recombinant protein was purified by passing through a Ni-NTA Superflow column (QIAGEN GmbH, Hilden, Germany) on a Biologic DuoFlow™ Chromatography System (Bio-Rad, CA, USA) according to the manufacturer's manual. The purified protein was analyzed by 12% SDS-PAGE.

With respect to western blot analysis, protein samples were subjected to electrophoresis on 12% SDS-PAGE gel and transferred onto nitrocellulose membrane using a Bio-Rad transblot apparatus (Bio-Rad, CA, USA). After overnight blocking in PBST mixed with 2% BSA, the membrane was incubated with goat anti-RSV antiserum (Meridian, Me., USA) at a dilution of 1:2000 for 2 h at 4° C. The membrane was washed three times with 150 mM PBST and then incubated with bovine anti-goat IgG-HRP conjugate (Santa Cruz, Calif., USA) in blocking buffer (PBST mixed with 2% BSA) at a dilution of 1:1000 for 1 h at RT Immune complexes were detected by the ECL method according to the manufacturer's instruction (GE Healthcare Europe, Uppsala, Sweden).

With respect to RSV stock preparation, the RSV Long strain was obtained from ATCC (catalog no. VR-26) and propagated in HEp-2 cells at 37° C. in a humidified atmosphere with 5% CO₂. The viruses were added to the cells in DMEM at a multiplicity of infection (m.o.i.) of 4:1. After 1 h, DMEM with 2% FCS was added to the flask and infection of cells was monitored for 3-4 days. RSV was harvested by centrifugation at 3,000×g at 4° C. to remove cellular debris, aliquoted, and stored at −80° C. until use.

Formalin-inactivated RSV (FI-RSV) was prepared as described by Kim et al. American Journal of Epidemiology 89: 422-434 (1969). Briefly, 1 part formalin (approximately 37%-40%) was incubated with 4,000 parts clarified virus lysate for 3 days at 37° C. and pelleted by centrifugation for 1 h at 50,000×g. The virus was diluted 1:25 in minimum essential medium (MEM) and subsequently precipitated with aluminum hydroxide (4 mg/ml), resuspended in 1/100 of the original volume in serum-free MEM, and stored at 4° C.

With respect to immunization and challenge of mice, plasmid DNA for immunization was isolated using the EndoFree Plasmid Giga kit (QIAGEN, Hilden, Germany) following the manufacturer's recommendations. Both plasmids and recombinant proteins were dissolved in PBS. For vaccination, the mice were randomly divided into groups of 9 animals per group as listed in Table 1. Mice were immunized intramuscularly with one of FI-RSV, plasmid, plasmid plus protein or saline solution on days 0, 14 and 28. The mice were bled before and after immunization at 2-week intervals. Mice were challenged intranasally with RSV (10⁶ TCID₅₀ in 50 μl) on day 14 after the last immunization. Mice were weighed daily following the challenge. The data were expressed as the percentages of the base weights at day 0 of challenge. Mice were sacrificed 5 days later and assayed for lung virus titers and leukocyte infiltration in bronchoalveolar fluids.

TABLE 1
Immunization groups

| Group | Vaccine |
|---|---|
| 1 | Naïve |
| 2 | FI-RSV |
| 3 | 100 μl saline solution |
| 4 | 100 μg proVAX/G |
| 5 | 100 μg His-G |
| 6 | 100 μg proVAX + 100 μg His-G |
| 7 | 100 μg proVAX/G + 100 μg OVA |
| 8 | 100 μg proVAX/G + 100 μg His-G |
| 9 | 100 μg proVAX/F |
| 10 | 100 μg His-F |
| 11 | 100 μg proVAX + 100 μg His-F |
| 12 | 100 μg proVAX/F + 100 μg OVA |
| 13 | 100 μg proVAX/F + 100 μg His-F |

With respect to the assay of antibodies, antibodies against virus were assayed by enzyme-linked immunosorbent assay (ELISA). The 96-well microtiter plates were coated overnight with UV-inactivated RSV in 0.05 M bicarbonate buffer (pH 9.6), 100 μl per well at 4° C. The plates were blocked with 5% BSA-PBST at 37° C. for 1 h, washed, and incubated with serially diluted serum for 1 h at 37° C. The secondary goat anti-mouse IgG conjugated with horseradish peroxidase (Bio-Rad Laboratories, Hercules, Calif., USA) was diluted 1:1000, added to each well and incubated at 37° C. for 1 h. Ten milligrams of TMB tablet was dissolved in 0.025 M phosphate-citrate buffer and added to each well for color development. After addition of 2 M H₂SO₄ the plate was read with a plate reader (Magellan; Tecan Group, Maennedorf, Switzerland) at 450 nm. Antibody titers were expressed as an absolute ratio of post/naive serum at a cutoff of 2.1.

With respect to the virus neutralization assay, the virus neutralization assay was performed as previously described with modifications (Singh et al. Vaccine 25: 6211-6223 (2007); Anderson et al. J Clin Microbiol 22: 1050-1052 (1985)). In brief, sera were serially diluted two-fold in a total of 100 μl PBS, heat inactivated at 56° C. for 30 min and incubated with 3×10³ TCID₅₀ of virus for 2 h at 4° C. Approximately, 5×10⁴ HEp-2 cells in 100 μl DMEM supplemented with 2% FCS were added to each well of a 96-well microtiter plate. The virus-serum mixture was added to the appropriate wells and incubated for 3 days in a 5% CO₂ incubator at 37° C. Plates were then washed three times with 0.05% Tween-20 in PBS and fixed with 80% cold acetone in PBS followed by blocking with 3% blocking buffer. Goat anti-RSV antibody (Meridian, Me., USA) was added to the appropriate wells and incubated for 60 min at 37° C. After three washings, bovine anti-goat IgG-HRP (Santa Cruz, Calif., USA) was added, the enzymatic reaction was developed from the average ODs were read at 450 nm/630 nm. The neutralization titer was calculated from the average OD of the wells by extrapolating the inverse of the serum dilution that resulted in 50% reduction of RSV activity.

With respect to virus quantification, lungs were harvested from immunized mice on day 5 post-RSV challenge, homogenized and then centrifuged at 2000 rpm for 10 min. Cell-free supernatants from these samples were snap-frozen in liquid nitrogen and virus was titrated in thawed samples. Briefly, serial log₁₀ dilutions of each test sample in 2% FCS-DMEM were added to microtiter plates containing 3×10³ HEp-2 cells/well and incubated at 37° C. with 5% CO₂. On Day 5, the wells were scored by visual inspection for the formation of syncytia. Endpoints were calculated by Karber's method. The amount of virus present in each suspension was expressed as the geometric mean virus titer (GMT; log₁₀ TCID₅₀/gram).

With respect to flow cytometric analysis, T cells were isolated and stained with phycoerythrin-(PE-), fluorescein isothiocyanate-(FITC-) or allophycocyanin-(APC-) conjugated mAbs in PBS for 30 min at 4° C. For intracellular staining, T cells were stimulated in vitro with G protein (10 μg/ml) for 8 h and subsequently treated with monensin (100 μg/ml) for 2 h. The cells were blocked with Fc-Block (BD Pharmingen, San Diego, USA) in PBS for 30 min at 4° C. before being fixed with 4% paraformaldehyde and permeabilized with saponin. The cells were intracellularly stained for 30 min at 4° C. with the appropriate concentrations of antibodies, including APC-labeled anti-FoxP3 or PECy5-labeled anti-CD25 antibody, FITC-labeled anti-CD4 antibody or PE-labeled anti-IL-10 antibody (BD Pharmingen, San Diego, USA). The cells were washed and analyzed with a FACScalibur using the Cell QuestPro Software (BD Bioscience, San Jose, USA).

With respect to the T cell proliferation assay, spleens were removed from immunized mice on day 7 after the last immunization and used to prepare single T cell suspensions (Tim. Journal of Immunological Methods 65: 55-63 (1983)). Single lymphocyte suspensions were incubated in triplicate in 96-well plates at 5×10⁴ cells/well, in RPMI-1640 plus 5% FCS at 37° C. in a 5% CO₂ incubator and stimulated for 48 h with 0.1 μg/ml of phorbol myristate acetate (PMA) plus 1 μg/ml Ionomycin (Ion) as positive control, 10 μg/ml UV-irradiated RSV antigen as specific antigen, 2 μg/ml bovine serum albumin (BSA) as irrelevant antigen, or no antigen as negative control. T cell proliferation was evaluated using the MTT method and OD values of the plates were read at 570 nm by a plate reader (Magellan; Tecan Austria GmbH) after 4 h of incubation with MTT (Wang et al. Vaccine 18: 1227-1235 (2000)). Data were expressed as the stimulation index (SI), calculated as the mean reading of triplicate wells stimulated with an antigen, divided by the mean reading of triplicate wells stimulated with medium.

With respect to cell isolation and adoptive transfer, single splenocyte suspensions were prepared from mouse spleen. CD4⁺CD25⁺ or CD4⁺CD25⁻ T cells were isolated and purified by using the MagCellect Mouse CD4⁺CD25⁺+T Cell Isolation Kit according to the manufacturer's protocol (R&D Systems, Inc., Minneapolis, Minn., USA). The resulting, CD4⁺CD25⁺, and CD4⁺CD25⁻ T cell suspensions were >90% pure as determined by flow cytometry (FACSCalibur, BD Bioscience, San Jose, USA). The CD4⁺CD25⁻ at 4×10⁵/mouse (about 6×10⁴ iTreg cells) or CD4⁺CD25⁺ at 2×10⁵/mouse (about 1.8×10⁵ nTreg cells) were adoptively transferred intravenously into mice.

With respect to the mixed lymphocyte reaction, the CD4⁺CD25⁺ or CD4⁺CD25⁻ T cell populations were purified from Balb/c mice as described under cell isolation above and used as the responder cells. Stimulator cells were isolated from the spleen of naïve C57BL/6 mice by panning using anti-CD3 monoclonal antibody (eBioscience, CA, USA) to delete T cells and were further treated with mitomycin C before use. The responder and stimulator cells were mixed at various ratios, from 1:1, 1:3 to 1:10 (Balb/c responder:C57BL/6 stimulator), seeded at 2×10⁵ total cells per well in triplicate wells, and cultured for 48 h. The response was measured by the MTS/PMS colorimetric assay (Kang et al. Vaccine 23: 5543-5550 (2005)) using OD values read at 490 nm on a plate reader (Magellan; Tecan Group, Maennedorf, Switzerland).

With respect to real-time PCR, total mRNA was extracted from lungs samples that were isolated from immunized mice on day 5 post-RSV challenge. cDNA was synthesized by reverse-transcription PCR and SYBR Green incorporation during quantitative Real-time PCR was measured using a Fast Start SYBR Green mix (Roche) in the ABI7400 Sequence Detection System (Applied Biosystems). The primers used are listed in Table 2.

TABLE 2
Real-time PCR primers (5′-3′)

| Target | | |
|---|---|---|
| genes | Forward | Reverse |
| IL-4 | ACAGGAGAAGGGACG | GAAGCCCTACAGACGA |
| | CCAT | GCTCA |
| | (SEQ ID NO: 9) | (SEQ ID NO: 10) |
| IL-5 | AGCACAGTGGTGAAAGAGA | TCCAATGCATAGCTGGT |
| | CCTT | GATTT |
| | (SEQ ID NO: 11) | (SEQ ID NO: 12) |
| IL-13 | GGAGCTGAGCAACATC | GGTCCTGTAGATGGCA |
| | ACACA | TTGCA |
| | (SEQ ID NO: 13) | (SEQ ID NO: 14) |
| IFN-γ | TCAAGTGGCATAGATGTG | TGGCTCTGCAGGATTT |
| | GAAGAA | TCATG |
| | (SEQ ID NO: 15) | (SEQ ID NO: 16) |
| HPRT | CTGGTGAAAAGGACC | TGAAGTACTCATTATA |
| | TCTCG | GTCAAGGGCA |
| | (SEQ ID NO: 17) | (SEQ ID NO: 18) |

With respect to plethysmography, measurements of respiratory system dynamic resistance (Rrs) and compliance (Cldyn) were measured by placing mice in a whole-body plethysmograph (model AniRes2005; Beijing Bestlab Technology Co. Beijing, China) according to the manufacturer's manual. In brief, mice were anaesthetized 5 days after RSV challenge. Tracheotomy was performed and the trachea connected to the ventilator. Mechanical ventilation was carried out, dynamic airway pressure (ΔP) and volume of chamber (ΔV) were recorded, and peak resistance and compliance were automatically measured as Rrs=ΔP/(ΔV/ΔT) and Clydn=ΔV/ΔP after each 200 μl intrajugular administration of various doses of acetylcholine chloride (mg) (Jin et al. European Journal of Immunology 38: 2451-2463 (2008)). With respect to the bronchoalveolar lavage (BAL) collection, five days after challenge, the mice were euthanized, a tracheotomy was performed, and the large airways were washed with 1 ml PBS containing 0.1% bovine serum albumin. The bronchoalveolar lavage (BAL) wash was centrifuged and the supernatant was removed. The BAL pellet was resuspended and the total cell count obtained by FACS. Cells were stained with Wright's Giemsa (Lichen Biotechnology Co., Ltd., Shanghai, China), and cell types were identified by morphological criteria. At least two hundred total cells were examined per slide for a differential count.

With respect to histopathology, lung tissues were fixed in buffered formalin, and transverse sections (thickness, 5 μm) were stained with hematoxylin and eosin. The histopathologic score (HPS) was based on grading of five different parameters: (i) peribronchiolar and bronchial infiltrates, (ii) bronchiolar and bronchial luminal exudates, (iii) perivascular infiltrates, (iv) amount of monocytes, and (v) parenchymal pneumonia. The HPS system assigned values from 0 to 21; the higher the score, the greater the inflammatory changes in the lung (Jafri et al. Journal of Infectious Diseases 189: 1856-1865 (2004); Mejias et al. Antimicrobial Agents and Chemotherapy 48: 1811-1822 (2004)). The HPS was determined by a pathologist who was unaware of the infection status of the animals from which specimens were taken.

With respect to statistical analysis, results are presented as means±standard error of the mean (S.E.M.). Student's t and non-parametric test analysis were used for data analysis. A value of p<0.05 was considered to be statistically significant.

### Example 2

### Expression of DNA and Protein Vaccines

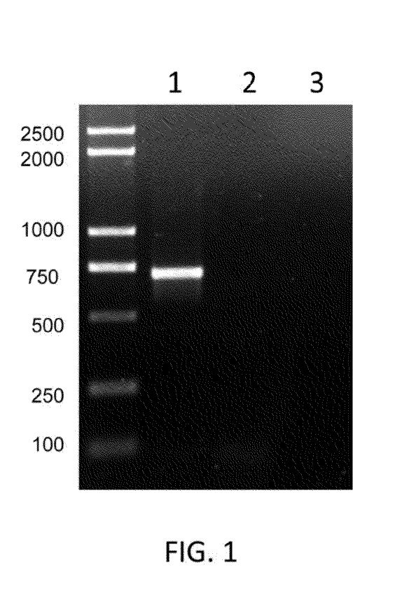

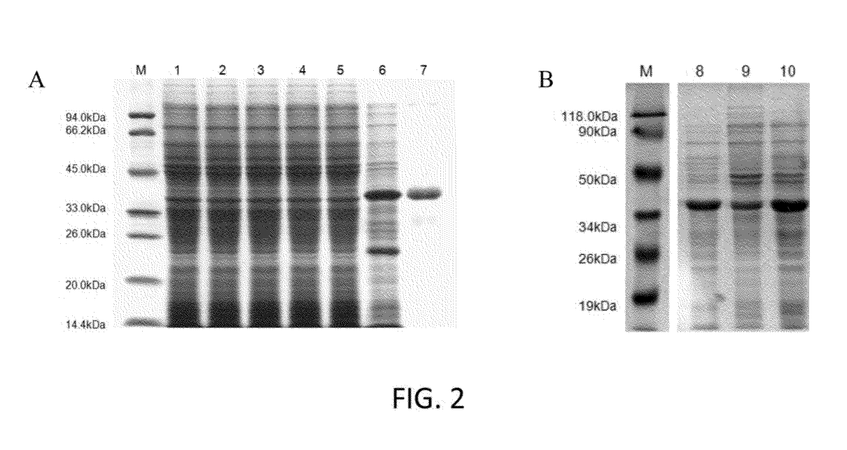

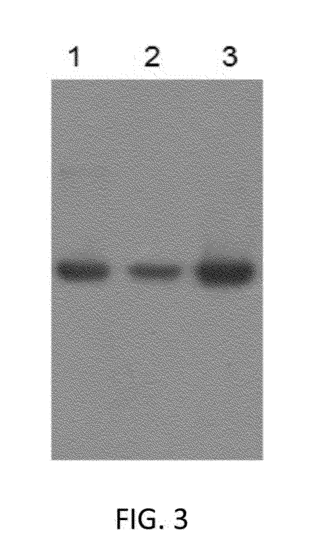

The eukaryotic expression constructs proVAX/G and proVAX/F were separately transfected into NIH/3T3 cells to confirm expression. Assay of G-specific mRNA by RT-PCR confirmed efficient expression (FIG. 1). The prokaryotic expression constructs pET28a(+)/G and pET28a(+)/F were transformed into E. coli BL21 (DE3) and the resultant recombinant protein was purified by Ni-NTA Superflow on a Biologic DuoFlow™ Chromatography System (pET28a(+)/G shown in FIGS. 2A-2B). The identity of the G protein and F protein produced was confirmed by Western blot using specific goat anti-RSV polyclonal antisera (the identity of the G protein shown in FIG. 3).

### Example 3

### Antibody Responses

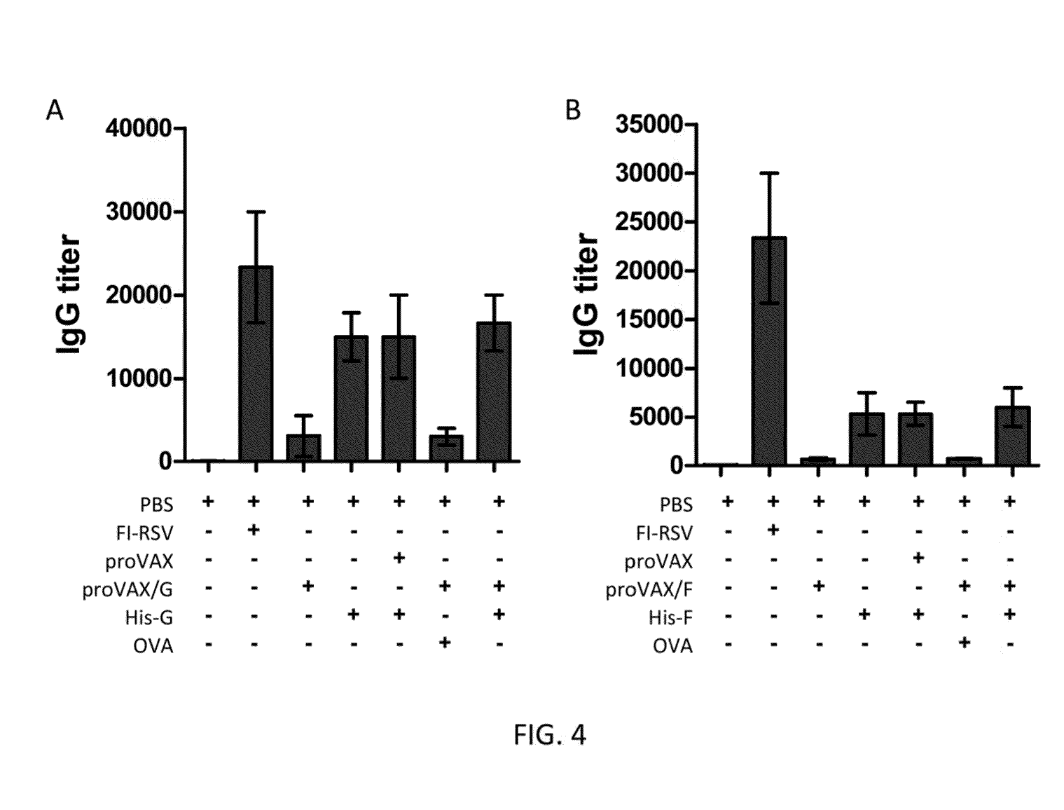

Groups of seven Balb/c mice were immunized intramuscularly on days 0, 14, and 28 and the level of IgG antibody against virus was determined by ELISA 7 days after the last immunization. proVAX/G and His-G given separately were compared with proVAX/G plus His-G protein and with FI-RSV as a positive control. In addition, proVAX/F and His-F given separately were compared with proVAX/F plus His-F protein and with FI-RSV as a positive control. Additional control mice received proVAX (empty vector) plus His-G, proVAX (empty vector) plus His-F, proVAX/G plus an irrelevant protein (ovalbumin; OVA), proVAX/F plus an irrelevant protein (ovalbumin; OVA) or PBS. All immunized groups produced detectable IgG antibodies (positive: ODExp/ODPre>2.1) except mice immunized with PBS. Although the highest level was achieved with FI-RSV immunized mice, groups immunized with proVAX/G+His-G, His-G alone or His-G+proVAX produced similar levels of antibody response. Groups immunized with proVAX/F+His-F, His-F alone or His-F+proVAX also produced an antibody response. The lowest levels were produced by proVAX/G, proVAX/G+OVA, proVAX/F or proVAX/F+OVA groups (FIGS. 4A-4B).

Studies in animals and humans had demonstrated that a higher level of neutralizing antibody correlated with a higher level of protection against RSV infection. Therefore the capability of the serum antibodies to neutralize RSV was assessed. The serum neutralization titer was determined as follows: pooled sera collected from immunized mice on day 7 after the last immunization were used to determine the RSV neutralization titer as described in Example 1. The lung RSV titer was determined as follows: lung samples from mice immunized with the indicated antigens and subsequently challenged with RSV (10⁶ TCID₅₀) were used to collect RSV. Viral titer was determined as described in Example 1 and discussed in Example 5. Data are presented from at least six replicates with standard error (Table 3). As shown in Table 3, the positive control, FI-RSV vaccination, provided the highest neutralizing antibody level; whereas antisera from His-G, proVAX+His-G, proVAX/G+His-G, His-F, proVAX+His-F, and proVAX/F+His-F immunized groups exhibited significant higher and similar levels of neutralizing antibodies. Thus, the level of binding antibody was approximately paralleled by the neutralizing activity.

TABLE 3
Serum neutralization titer and lung RSV titer (log₁₀)

| | Serum neutralization | Virus titer (log₁₀TCID₅₀/gram |
|---|---|---|
| Antigen | titer | of lung tissue) |
| PBS | 0.418 ± 0.052 | 5.107 ± 0.0994 |
| FI-RSV | 3.710 ± 0.036 | 1.543 ± 0.1233 |
| proVAX/G | 1.867 ± 0.089 | 3.790 ± 0.2479 |
| proVAX/F | 1.667 ± 0.032 | 4.893 ± 0.3034 |
| His-G | 3.354 ± 0.026 | 2.217 ± 0.3005 |
| His-F | 2.911 ± 0.023 | 3.783 ± 0.1590 |
| proVAX/G + His-G | 3.463 ± 0.042 | 1.850 ± 0.1041 |
| proVAX/F + His-F | 3.089 ± 0.030 | 3.857 ± 0.2942 |
| proVAX + His-G | 3.454 ± 0.038 | 2.023 ± 0.1468 |
| proVAX + His-F | 3.085 ± 0.050 | 3.883 ± 0.2205 |
| proVAX/G + OVA | 1.935 ± 0.046 | 3.300 ± 0.2887 |
| proVAX/F + OVA | 1.634 ± 0.032 | 4.733 ± 0.2333 |

### Example 4

### Suppression of Auto-Reactive CD4⁺ T Cell by Co-Immunization with Antigen-Matched DNA and Protein Vaccines

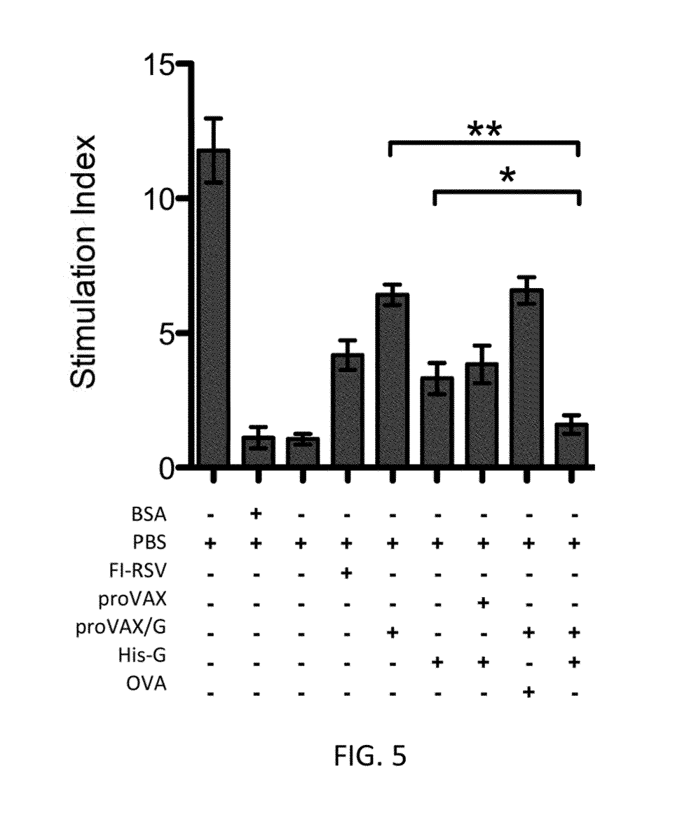

Since severe VID upon RSV challenge is mediated by induction of autoreactive CD4⁺ T cells and co-immunization with antigen-matched DNA and protein vaccines has been shown to impair antigen-specific T cell and not antibody responses, T cell proliferative responses to UV-irradiated RSV antigen in vitro 7 days after the last immunizations were examined. The lowest level of proliferative response was observed in T cells isolated from mice co-immunized with proVAX/G+His-G, whereas strong proliferative responses were observed in the T cells isolated from the other immunized groups (FIG. 5) except for the PBS or BSA negative controls. The strong proliferative responses were observed in the T cells isolated from proVAX/G immunized group, showed that the cellular immune response was well elicited in the group. Co-immunizations with either proVAX/G+OVA, or His-G+proVAX vector as antigen-mismatched controls did not reveal any unrelated vector or protein influences on the response. This result shows that co-immunization with the DNA vaccine plus protein, proVAX/G+His-G, was unique in preferentially suppressing the T cell proliferative response.

### Example 5

### Protection Measured as Reduction of Viral Load after RSV Challenge

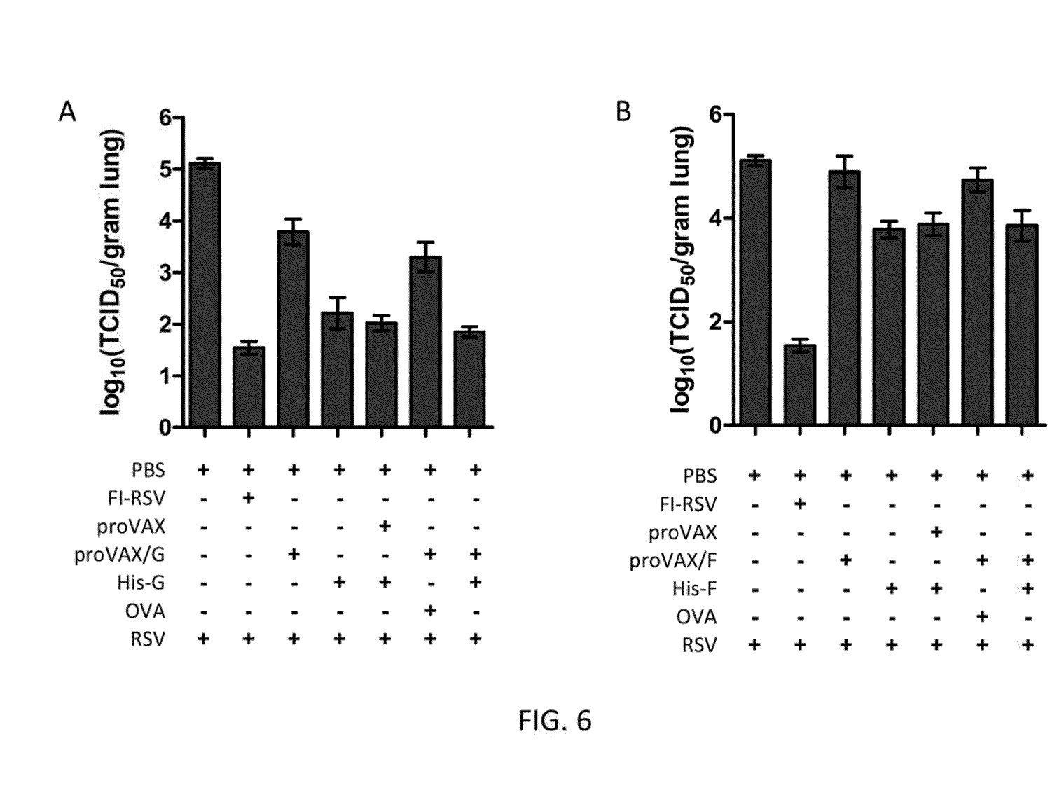

Two weeks following the final immunization, mice were challenged with RSV intranasally with 10⁶ TCID₅₀ (50 μl) per animal (see Table 3 and FIGS. 6A-6B). Virus titers were quantified in lung samples and in BALs collected 5 days after the challenge when the viral load peaked in the lungs. RSV replicated to significant titers at 5.107±0.0994 log₁₀ TCID₅₀/gram of lung tissue in PBS control mice. Mice immunized with FI-RSV, His-G, proVAX+His-G, or proVAX/G+His-G exhibited dramatic reductions of viral titer compared to the PBS, proVAX/G and proVAX/G+OVA immunized groups (Table 3 and FIG. 6A). Mice immunized with His-F, proVAX+His-F, or proVAX/F+His-F also exhibited reductions of viral titer compared to the PBS, proVAX/F and proVAX/F+OVA immunized groups (Table 3 and FIG. 6B). The protection was apparently correlated with neutralizing antibody titers (Table 3).

### Example 6

### Elimination of VID from the Co-Immunized Mice

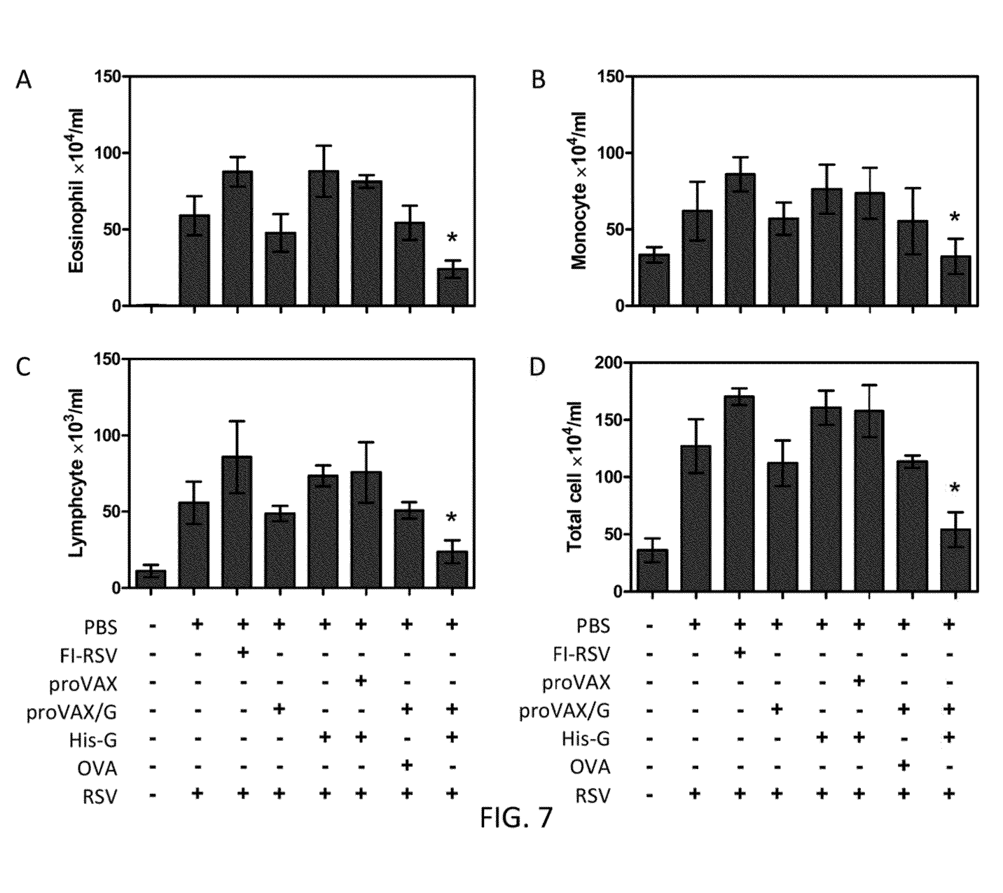

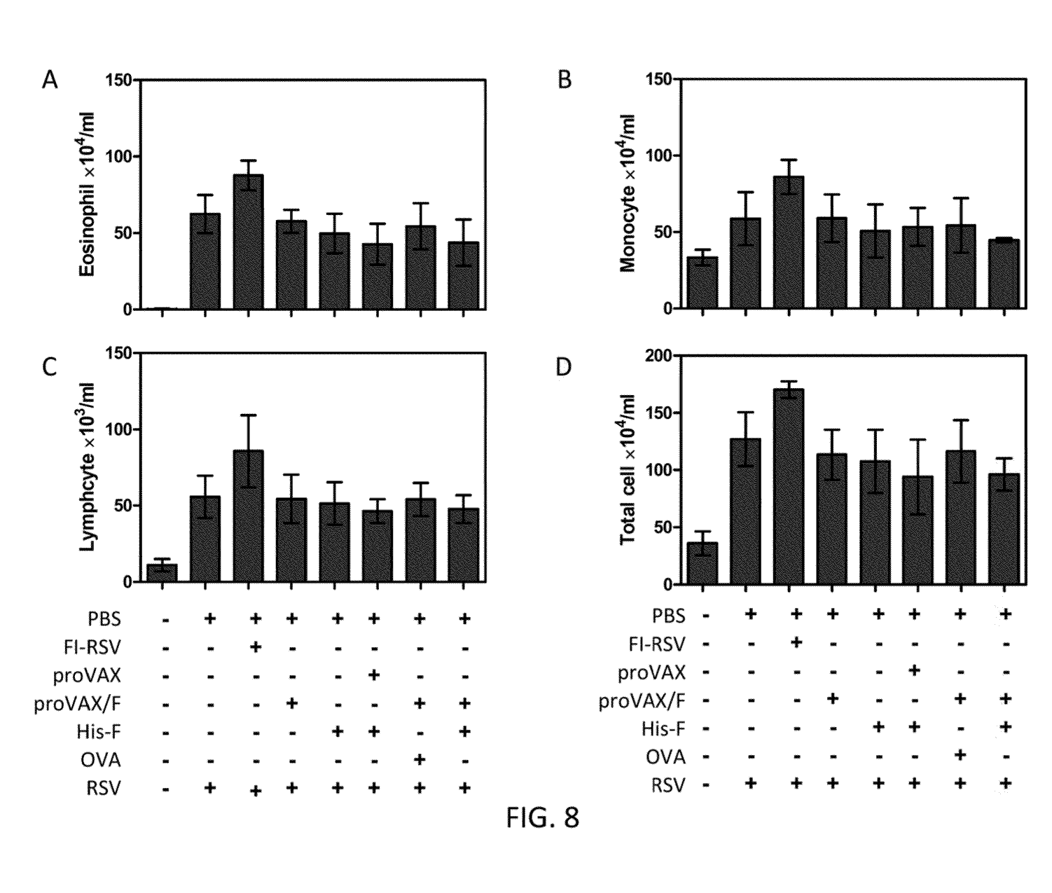

The ability of the co-immunization strategy to protect animals from airway hyper-responsiveness (AHR) after RSV challenge was examined. Cells were isolated from BALs, stained, and analyzed by microscopy to measure the amount of eosinophils, lymphocytes and monocytes present after RSV challenge. As shown in FIGS. 7A-7D, the infiltration of inflammatory cells was significantly less in the proVAX/G+His-G co-immunized group compared with the other immunized groups, indicating that co-immunization with RSV G antigens protects mice from RSV infection and ameliorates pulmonary inflammatory response. Similarly, the infiltration of inflammatory cells was less in proVAX/F+His-F co-immunized group compared with the other immununized group. (FIGS. 8A-8D). The lungs of groups immunized with FI-RSV or His-G or proVAX+His-G exhibited massive infiltrations after the RSV challenge.

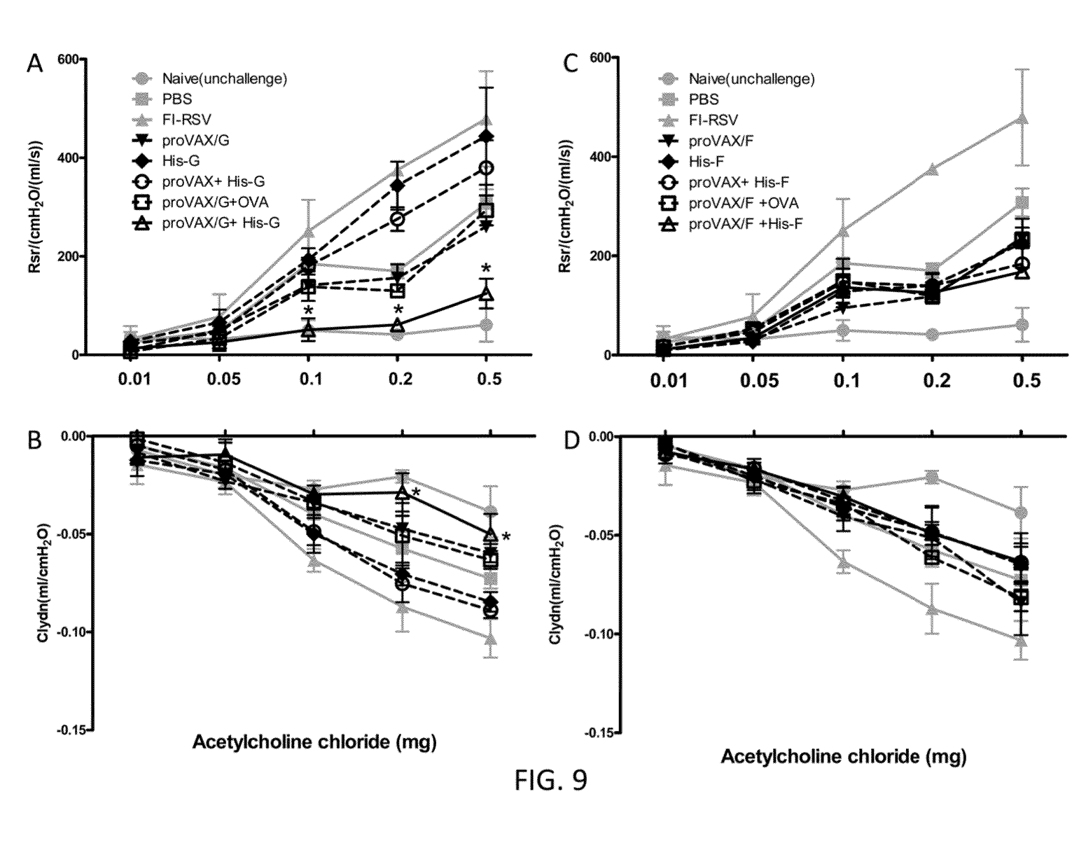

Due to the infiltration, RSV infected mice developed significant airway obstruction. To assess this degree of airway obstructions among the groups, whole-body plethysmograpy was performed during stimulation with various doses of acetylcholine chloride by intra jugularadministration five days after RSV challenge. As shown in FIGS. 9A-9B, the least values of Rrs and Cldyn in response to the acetylcholine stimulation were seen in the proVAX/G+His-G co-immunized group; whereas, Rrs and Cldyn values were significantly increased in the mice immunized with FI-RSV or His-G or proVAX+His-G compared with PBS controls. This result showed that airway obstruction was ameliorated by the proVAX/G+His-G co-immunizations. Similar results were seen for proVAX/F+His-F co-immunized group (FIGS. 9C-9D).

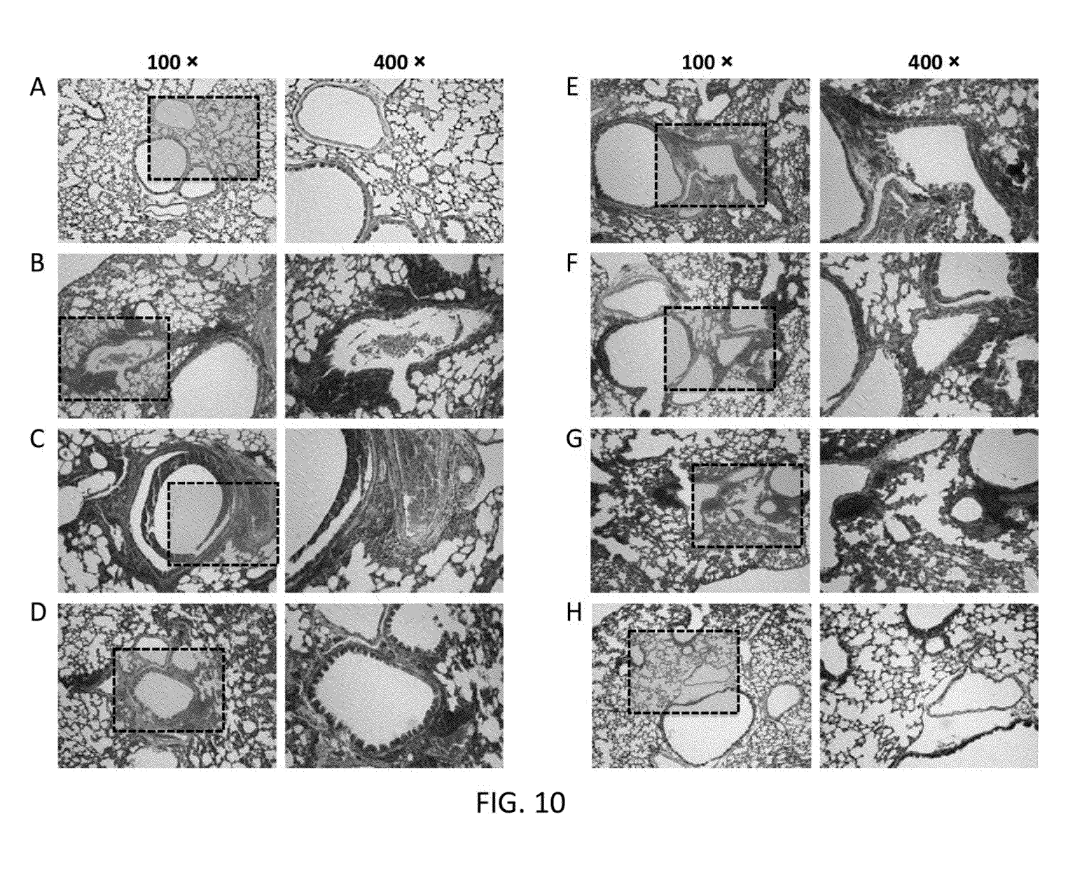

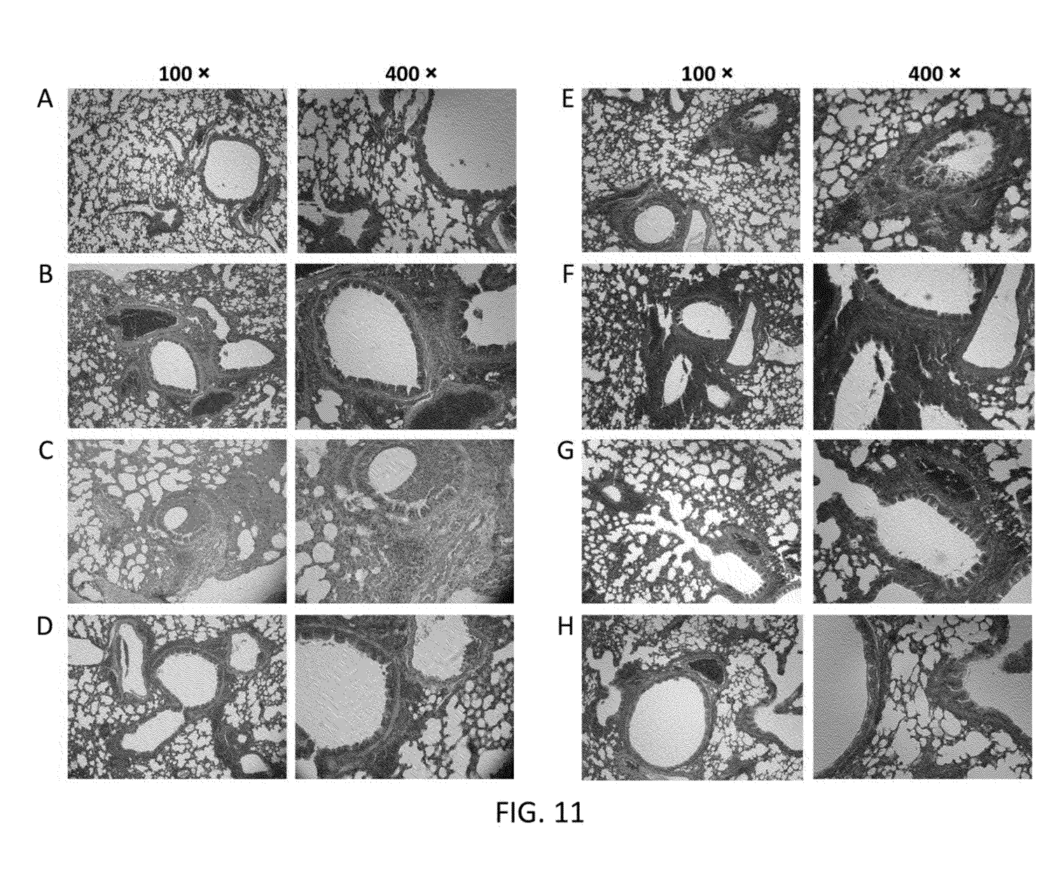

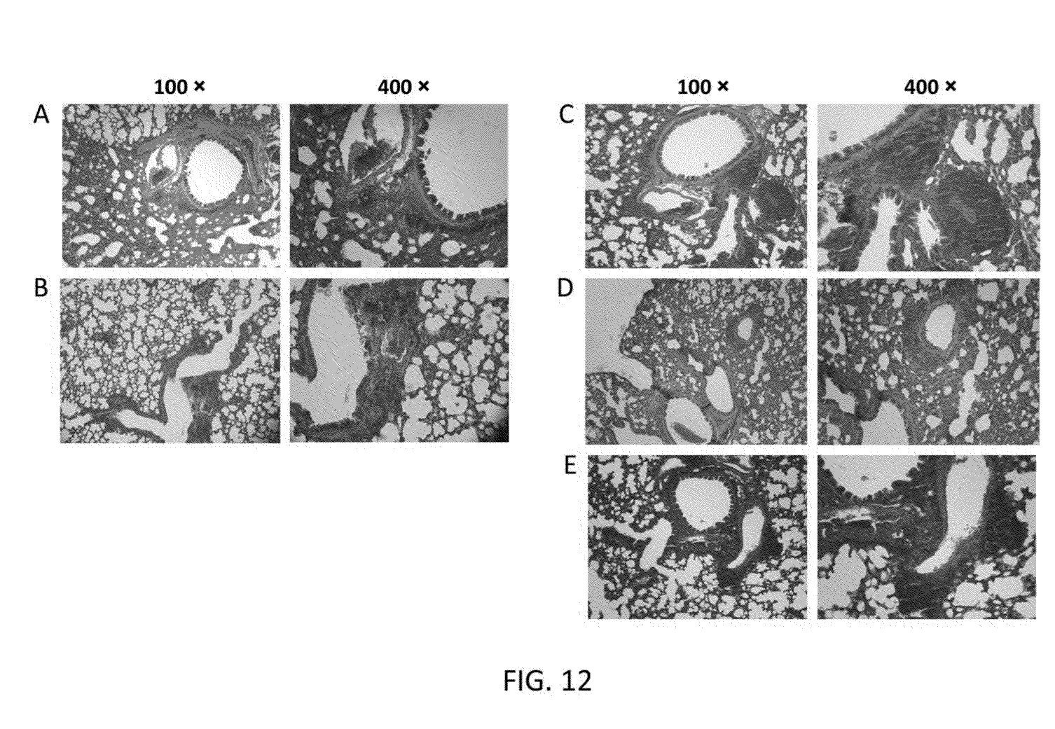

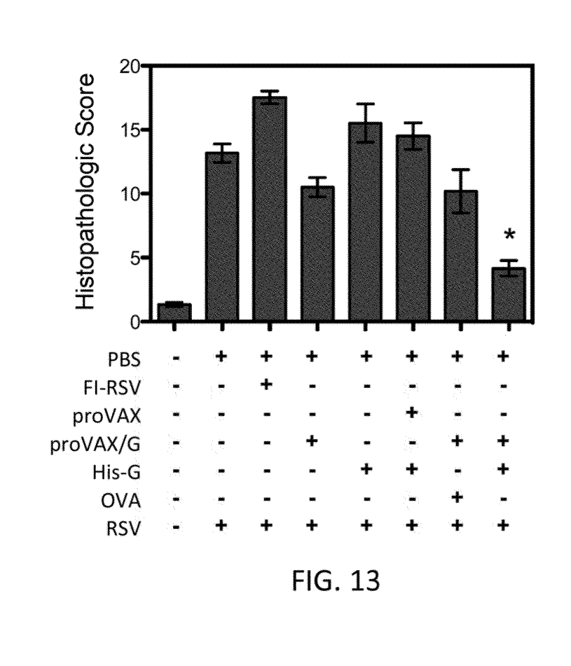

The amelioration was further assessed via the histopathological analysis of lung sections. The PBS control group displayed histopathological features of RSV infection 5 days post-challenge (FIGS. 10A-10H, FIGS. 11A-11H, and FIGS. 12A-12E). RSV replication resulted in marked lung inflammation with a dense lymphocytic infiltrate. The intense lymphocytic infiltration was noted in both the perivascular and peribronchial areas. Compared with mice immunized with proVAX/G, proVAX/G+OVA or PBS, mice immunized with FI-RSV or His-G or proVAX+His-G gave significantly higher histopathologic scores (HPS) (FIG. 13), with dense peribronchial and perivascular circumferential infiltrates and severe pneumonia (FIGS. 10A-10H). However, mice immunized with proVAX/G+His-G showed significantly less pulmonary inflammatory response and lung HPS values were significantly lower than in the other groups. These results further demonstrated that co-immunization with proVAX/G+His-G or proVAX/F+His-F can ameliorate the pulmonary histopathogensis of RSV infection.

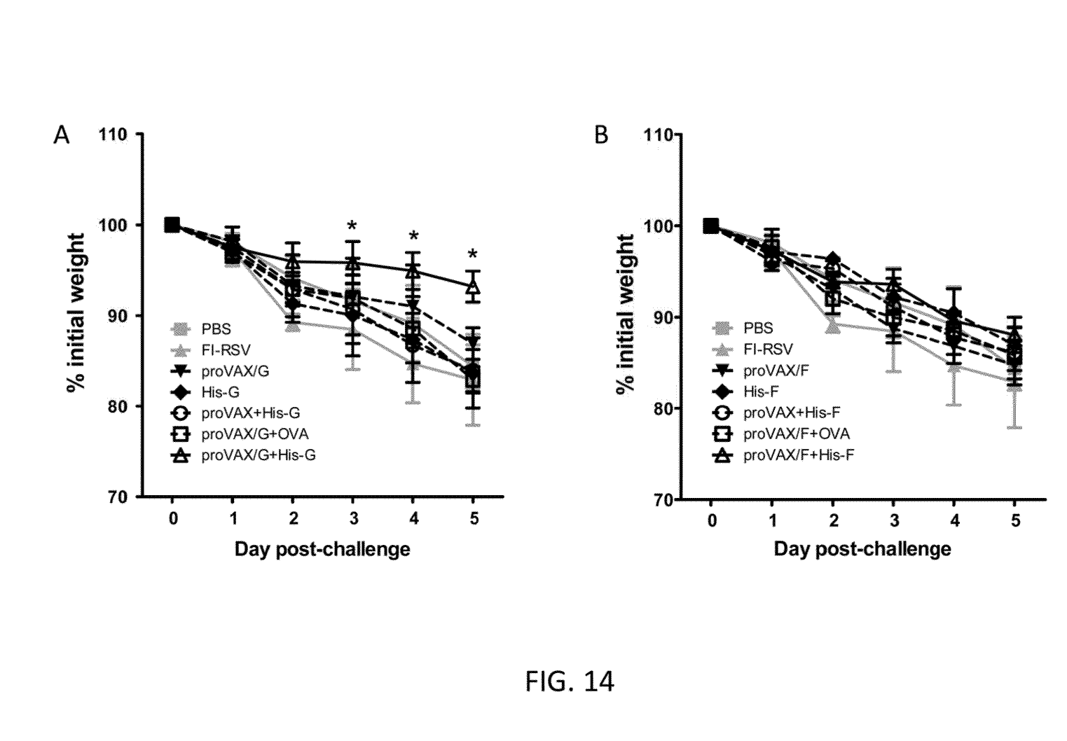

Besides impairing respiratory function, RSV infection can greatly affect body weight. After challenge with live RSV, the mice were weighed daily. All immunization groups had an early onset (day 2) of weight loss relative to the first day after challenge, however the proVAX/G+His-G co-immunized groups exhibited statistically less weight loss than other groups at days 5 to 7 postchallenge (FIG. 14A, p<0.05). In contrast, at days 8 to 9 after challenge, mice immunized with FI-RSV or His-G or proVAX+His-G had greater weight loss than other groups. Hence, co-immunization with proVAX/G+His-G can substantially reduce the severity of illness after live RSV challenge. Similar results were seen for proVAX/F+His-F co-immunized groups (FIG. 14B).

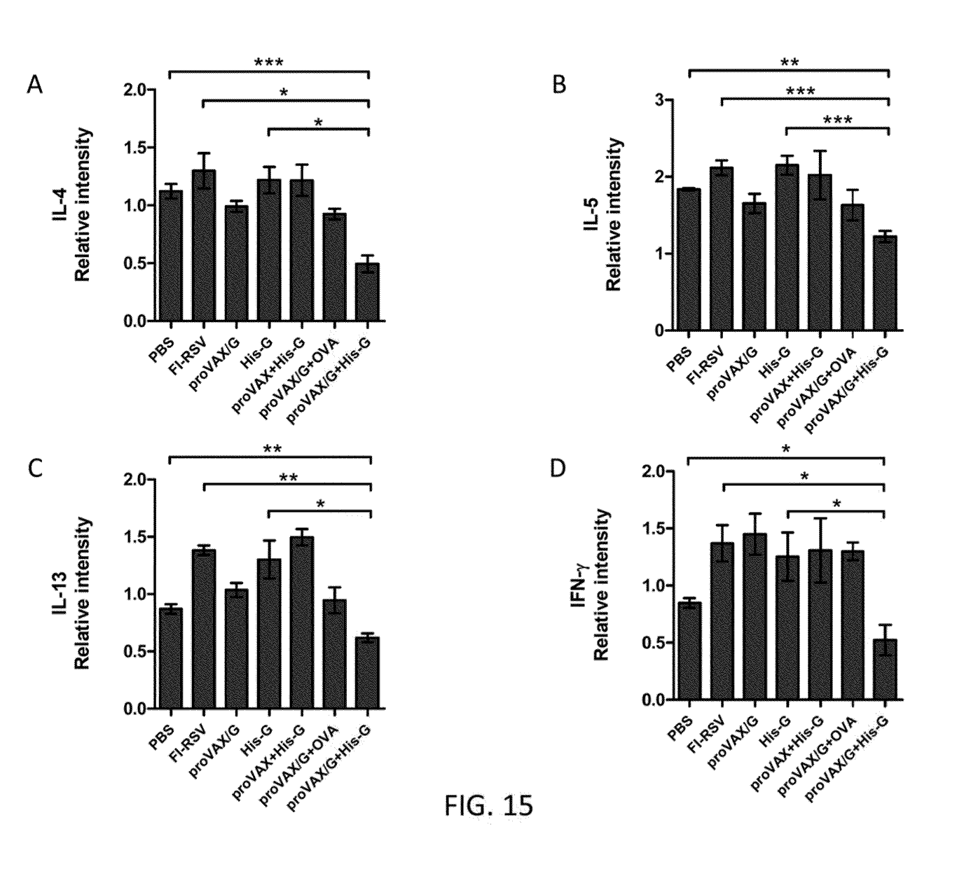

FI-RSV-induced VID was previously demonstrated to be associated with activation of a Th2 type response. To examine if cytokine profiles were affected by the co-immunizing regimens, qPCR was used to assay the levels of cytokines in lung tissues on day 5 after RSV challenge. As shown in FIGS. 15A-15D, the levels of nearly all cytokines tested in the His-G or proVAX+His-G-immunized groups were similar to those in the FI-RSV immunized group; whereas, mice co-immunized with proVAX/G+His-G produced the lowest levels of these cytokines. This shows that both Th1 and Th2 responses were suppressed by the co-immunization. Mice immunized with proVAX/G or proVAX/G+OVA showed higher levels of IFN-γ expression, suggesting that the DNA vaccination could elicit immunity polarized towards Th1.

### Example 7

### RSV-G Specific iTreg Cells Impair T Cell Responses

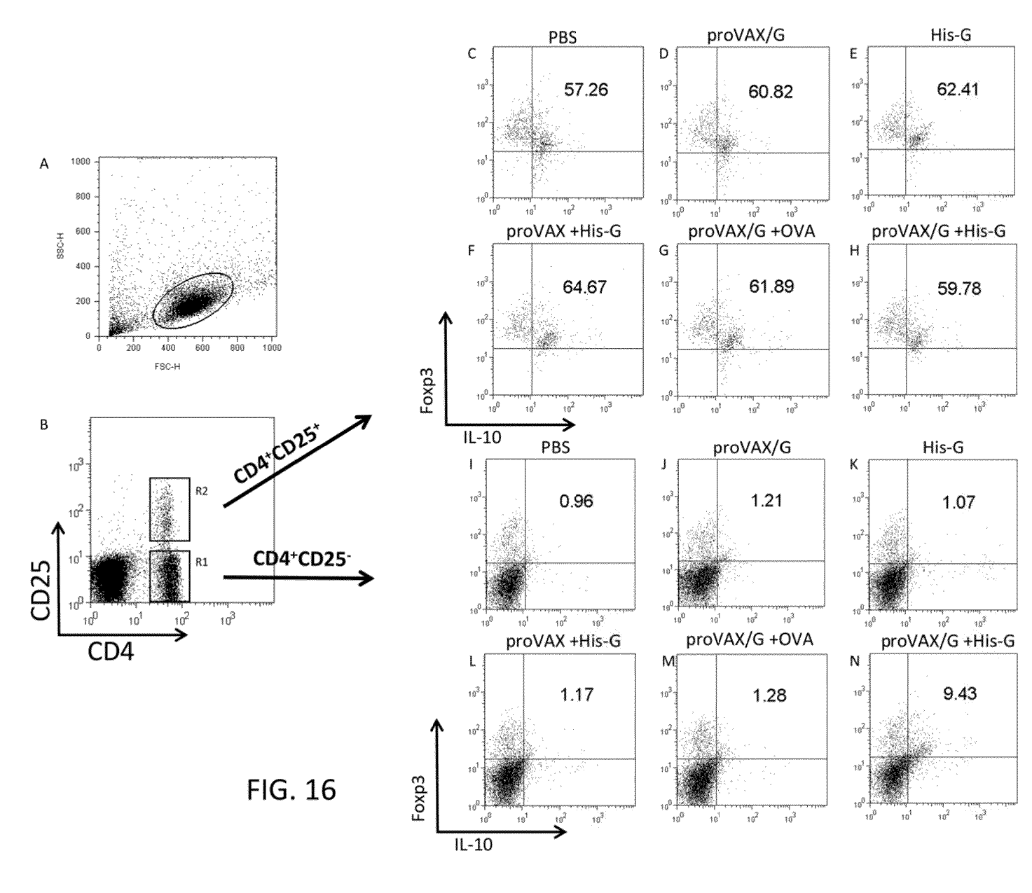

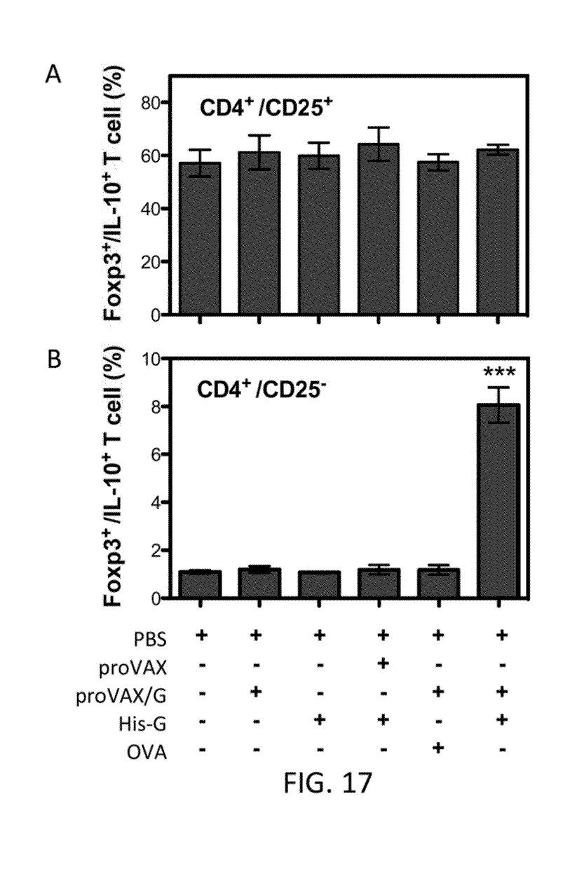

It was previously demonstrated that the impaired T cell response induced by co-immunization with DNA and protein vaccines was due to induction of CD4⁺CD25⁻ IL-10⁺FoxP3⁺ iTreg cells. To determine if the impaired T cell responses and skewed cytokine expressions observed here in the co-immunized group were due to the induction of iTreg cells, T cell phenotypes were analyzed by FACS 7 days after the last co-immunization. The results showed that the population of CD4⁺CD25⁻IL-10⁺FoxP3⁺ iTreg cells in spleen was indeed induced at a significantly higher level (p<0.001) in the mice co-immunized with proVAX/G+His-G compared with the other groups (FIGS. 16A-16N and FIGS. 17A-17B). In contrast, as a nTreg control, CD4⁺CD25⁺ T cells from each group highly expressed both FoxP3 and IL-10, but there was no significant difference in their concentration between the groups.

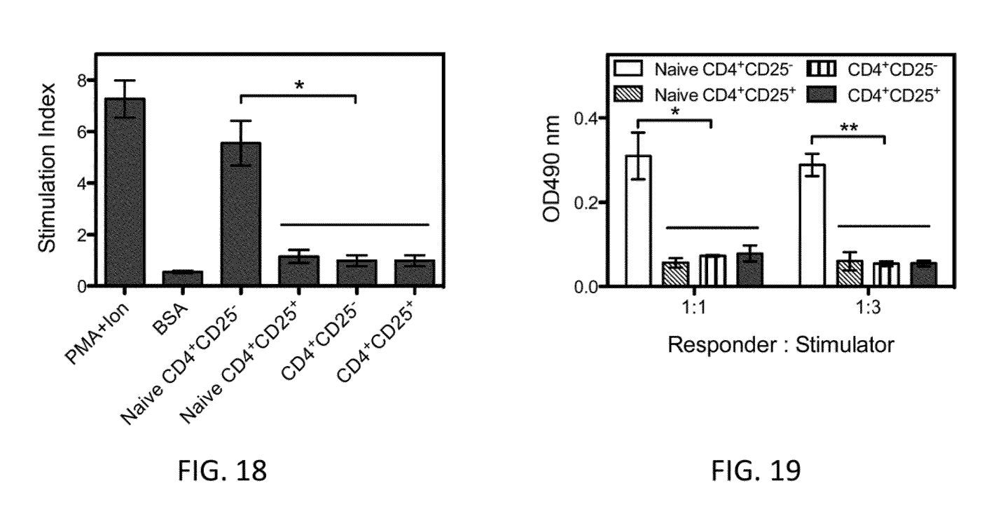

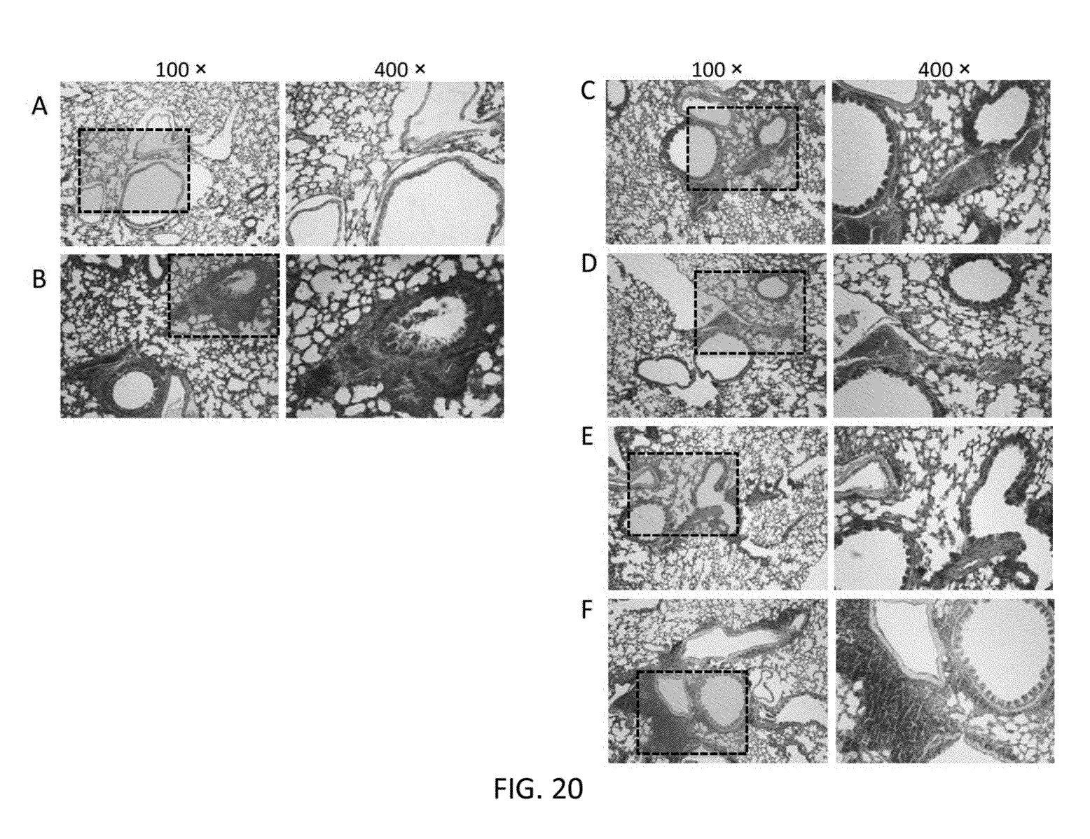

CD4⁺CD25⁻ iTreg cells were adoptively transferred to test their role in the amelioration of VID observed in vivo. Naïve Balb/c mice received CD4⁺CD25⁻ iTreg cells obtained from co-immunized mice. Controls received CD4⁺CD25⁺ cells as a nTreg population prepared from naive mice. The recipient mice were then immunized against His-G antigen and their responses to this immunization were assessed in T cell proliferation assays and MLR in vitro. As shown in FIG. 18, splenocytes from mice that had received CD4⁺CD25⁻ iTreg cells isolated from Balb/c mice that had been co-immunized with proVAX/G+His-G significantly inhibited T responder cell-recalled immune responses. The same splenocytes also inhibited proliferation in response to allogeneic APCs (FIG. 19), while the corresponding CD4⁺CD25⁻ cells from naïve donors did not inhibit proliferation. Splenocytes from mice that had received CD4⁺CD25⁺ nTreg cells from immunized mice, and cells from those mice that had received CD4⁺CD25⁺ nTreg cells from naïve mice could suppress T cell proliferation equally well, suggesting a nonspecific suppression by the transferred nTreg cells. These results indicated that co-immunization with proVAX/G+His-G could induce the CD4⁺CD25⁻IL-10⁺FoxP3⁺ iTreg cells that impair T cell response in an antigen dependent manner.

### Example 8

### Adoptive Transfer of CD4⁺CD25⁻ T Cells Arrests Ag-Specific Inflammation

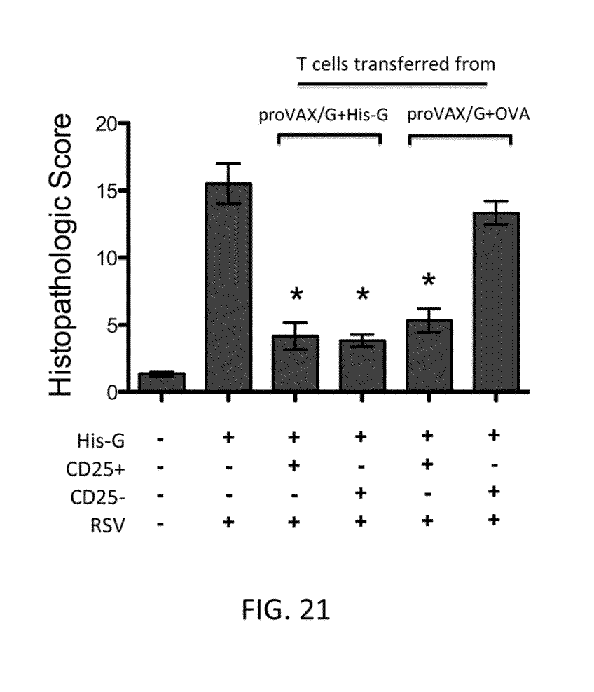

CD4⁺CD25⁻ iTreg and CD4⁺CD25⁺ nTreg cells were purified from mice co-immunized with proVAX/G+His-G or proVAX/G+OVA (antigen-mismatched control) and adoptively transferred intravenously into mice that had been previously immunized with His-G. The mice were challenged with RSV infection 7 days later. Lymphocytic infiltration was significantly decreased in mice receiving the CD4⁺CD25⁻ iTreg cells from the mice co-immunized with proVAX/G+His-G, but not in those receiving such cells from the proVAX/G+OVA immunized mice (FIGS. 20A-20F). Histological analysis revealed less inflammatory cell infiltrations in the groups received antigen specific CD4⁺CD25⁻ iTreg cells (FIGS. 20A-20F and FIG. 21). Although similar improvements of histopathological outcome were also observed in groups that received either CD4⁺CD25⁺ nTreg cells, the improvement from nTreg did not exhibit any antigen specificity.

CD4+CD25− cells from mice co-immunized with a DNA vaccine encoding G antigen and a G protein vaccine dramatically reduce the VID seen after live RSV challenge (FIGS. 20A-20F and FIG. 21). It is noted that CD4+CD25− cells transferred from mismatched vaccination regimens into His-G immunized mice resulted in a greater severity of clinical onset upon infection, suggesting that these cells from antigen-mismatched controls might suffer from a lack of iTreg cells and contain effector T cells (FIGS. 20A-20F and FIG. 21).

Co-immunization with an RSV G antigen-expressing DNA vaccine and its G protein together (proVAX/G+His-G) not only protected from RSV challenge in mice, but also suppressed the exacerbated pulmonary inflammation that leads to VID. The prevention of RSV infection and the reduced VID were apparently due to the induction of both high levels of antigen specific neutralizing antibody and of iTreg cells that could suppress T-cell recalled proliferation in an antigen-specific manner. Such co-immunization up-regulated the level of the anti-inflammatory cytokine IL-10, while down regulating the RSV-induced inflammatory cytokines, such as IL-4, IL-5, IL-13 and IFN-γ.

Although CD4+CD25+ cells (nTreg) from either co-immunized mice or mismatch controls were able to reduce inflammatory cell infiltrations in recipients after transfer (FIGS. 20A-20F and FIG. 21), they provided only a global nonspecific suppression. Presumably their suppression of inflammation is limited in vivo because nTreg is not an inducible population and has a plasticity dependent on immune macroenvironment. For example, it is difficult to induce substantial numbers of nTreg cells in vivo or ex vivo. Furthermore, a capacity for global immunosuppression might increase the risk of developing cancer and create an opportunity for infections

We claim:

1. A vaccine against respiratory syncytial virus (RSV) infection comprising an RSV antigenic peptide and a nucleic acid encoding the RSV antigenic peptide wherein the vaccine stimulates iTreg cells (CD4⁺, CD25⁻, FoxP3⁺, IL-10⁺).

2. The vaccine of claim 1, wherein the RSV antigenic peptide is selected from the group consisting of F glycoprotein, G glycoprotein, SEQ ID NO: 4 (optimized amino acid RSV G amino acid sequence), SEQ ID NO: 25 (optimized amino acid RSV F amino acid sequence) and functional fragments thereof.

3. The vaccine of claim 1, wherein the nucleic acid is DNA and the DNA encodes for the RSV antigenic peptide selected from the group consisting of F glycoprotein, G glycoprotein, SEQ ID NO: 4 (optimized amino acid RSV G amino acid sequence) SEQ ID NO: 25 (optimized amino acid RSV F amino acid sequence) and functional fragments thereof.

4. The vaccine of claim 3, wherein the DNA is present in a linear expression cassette of a circular plasmid.

5. The vaccine of claim 4, wherein the plasmid is selected from the group consisting of pVAX, pcDNA3.0, and proVAX.

6. The vaccine of claim 4, wherein the plasmid further comprises a promoter selected from the group consisting of CMV, SV40 early promoter, SV40 later promoter, metallothionein promoter, murine mammary tumor virus promoter, Rous sarcoma virus promoter, and polyhedrin promoter.

7. The vaccine of claim 1, wherein the nucleic acid and antigenic peptide are at a mass ratio selected from the group consisting of 5:1 and 1:5; and 1:1 and 2:1.

8. The vaccine of claim 1, wherein the vaccine is capable of being electroporated into a subject in need thereof.

9. A vaccination kit comprising a vaccine administration device and the vaccine of claim 1.

10. The kit of claim 9, wherein the vaccine administration device is selected from the group consisting of vaccine gun, needle, and an electroporation device.

11. The kit of claim 10, wherein the electroporation device is a minimally-invasive electroporation device.

12. A method for preventing or treating respiratory syncytial virus infection in a patient, the method comprising administering to a subject in need thereof the vaccine of claim 1.

13. The method of claim 12, wherein the subject is further protected from airway hyper-responsiveness (AHR) after respiratory syncytial virus challenge.

14. The method of claim 12, wherein the RSV antigenic peptide is selected from the group consisting of F glycoprotein, G glycoprotein, SEQ ID NO: 4 (optimized amino acid RSV G amino acid sequence) SEQ ID NO: 25 (optimized amino acid RSV F amino acid sequence) and functional fragments thereof.

15. The method of claim 12, wherein the vaccine is administered by electroporation.

16. A method of inducing neutralizing antibody against respiratory syncytial virus infection and suppressing inflammatory T cells, the method comprising administering the vaccine of claim 1 to a subject in need thereof.

17. The method of claim 17, wherein suppressing inflammatory T cell comprises inducing iTreg cells, and suppressing auto-reactive CD4+ and CD8+ T cells.

18. The method of claim 16, wherein the RSV antigenic peptide is selected from the group consisting of F glycoprotein, G glycoprotein, SEQ ID NO: 4 (optimized amino acid RSV G amino acid sequence), SEQ ID NO: 25 (optimized amino acid RSV F amino acid sequence) and functional fragments thereof.

19. A method of treating or preventing vaccine-induced disease in a subject immunized against respiratory syncytial virus (RSV), the method comprising administering the vaccine of claim 1 to a subject in need thereof.

20. The method of claim 19, wherein the subject is immunized with formalin-inactivated RSV (FI-RSV) or RSV antigen prior to encounter with natural RSV infection.

21. The method of claim 19, wherein the RSV antigenic peptide is selected from the group consisting of F glycoprotein, G glycoprotein, SEQ ID NO: 4 (optimized amino acid RSV G amino acid sequence), SEQ ID NO: 25 (optimized amino acid RSV F amino acid sequence) and functional fragments thereof.

22. The method of 19, wherein the vaccine is administered via electroporation.

23. The method of claim 19, wherein the electroporation route is selected from the group consisting of intradermal and intramuscular.

24. The method of claim 19, wherein the vaccine is electroporated with a minimally-invasive electroporation device.


SEQUENCE LISTING (54 sequences)

SEQ ID NO: 1 - protein, 574 aa, Human respiratory syncytial virus
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 2 - protein, 298 aa, Human respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMETFHSTSSEGNLSPSQVSTTSEHPSQPSSPPNTTRQ

SEQ ID NO: 3 - DNA, 714 bp, Artificial Sequence
  Synthetic Oligonucleotide
gaattcatgcataaggtgactcctacaacggctatcattcaggacgccacctcccaaatcaaaaacactacacccacttatctgacacagaacccccaactgggcatcagcccttccaacccttctgaaatcacttcccagatcaccactatcttggcttctactacccctggggtcaagtccactctgcagtctaccacagtcaaaacaaagaatacaaccactacccagactcagccaagcaagccaacaacaaagcagcgacaaaataaaccccctagtaagccaaataacgacttccactttgaggtgtttaattttgttccttgcagtatctgctctaacaatcccacctgttgggcgatatgtaaacgcatcccgaataagaagccaggtaagaagacaaccacaaagcccacaaagaaacccaccctgaaaacaaccaagaaagatccaaagccccagacgaccaaaagcaaagaggtgcctacgacaaagccgacagaagagcctacaatcaataccaccaagaccaacattattaccacccttcttacttctaacactaccggaaatcctgagttgacaagtcagatggagacattccattcaacgtcctcagaaggcaacccaagcccctcccaggtatcaaccacctctgaatacccgagccagccctccagtcccccaaacaccccacggcagtaatctaga

SEQ ID NO: 4 - protein, 233 aa, Artificial Sequence
  Synthetic Peptide
MHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 5 - DNA, 714 bp, Artificial Sequence
  Synthetic Oligonucleotide
gaattcatgcataaagtaaccccgaccaccgctatcatccaggacgctaccagccagatcaaaaacactacgcctacctatctgactcagaacccgcaactgggcatctccccgtccaatccgtctgaaattacctcccagatcactaccatcctggcatccactactccgggtgtgaaatctaccctgcagtccactaccgtaaaaacgaaaaacaccaccactacccagactcagccttccaaacctactacgaaacagcgtcagaacaaaccgccgagcaaaccgaacaacgacttccactttgaagttttcaacttcgtcccatgcagcatttgtagcaacaatccgacctgctgggcaatttgcaaacgcatcccaaacaaaaagccgggcaaaaagacgaccactaaaccaaccaagaaacctaccctgaaaactaccaaaaaagacccgaaaccgcagaccaccaaatctaaagaagttccgacgaccaaaccgaccgaggaaccgacgatcaacaccacgaaaacgaacatcatcaccaccctgctgacctctaacactaccggtaatccggagctgactagccagatggaaacctttcacagcacttcttctgaaggtaacccatctccgagccaggtgtccaccacttctgaatacccgagccaaccgtcctccccgcctaatacgccgcgtcaataactcgag

SEQ ID NO: 6 - DNA, 15226 bp, Human respiratory syncytial virus
acgcgaaaaaatgcgtacaacaaacttgcgtaaaccaaaaaaatggggcaaataagaatttgataagtaccacttaaatttaactcccttggttagagatgggcagcaattcgttgagtatgataaaagttagattacaaaatttgtttgacaatgatgaagtagcattgttaaaaataacatgctatactgacaaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtgctacaaaatggaggttatatatgggaaatgatggaattaacacattgctctcaacctaatggtctaatagatgacaattgtgaaattaaattctccaaaaaactaagtgattcaacaatgaccaattatatgaatcaattatctgaattacttggatttgatcttaatccataaattataattaatatcaactagcaaatcaatgtcactagcaccattagttaatataaaacttaacagaagacaaaaatggggcaaataaatcaactcagccaacccaaccatggacacaacccacaatgataccacaccacaaagactgatgatcacagacatgagaccgttgtcacttgagactacaataacatcactaaccagagacatcataacacacagatttatatacttaataaatcatgaatgcatagtgagaaaacttgatgaaagacaggccacatttacattcctggtcaactatgaaatgaaactattgcacaaagtaggaagcactaaatataaaaaatatactgaatacaacacaaaatatggcactttccctatgccgatattcatcaatcatgatgggttcttagaatgcattggcattaagcctacaaagcatactcccataatatacaagtatgatctcaatccatgaatttcaacacaagattcacacaatccaaaacaacaactttatgcataactacactccatagtccaaatggagcctgaaaattatagtaatttaaaattaaggagagacataagatagaagatggggcaaatacaaagatggctcttagcaaagtcaagttgaatgatacactcaacaaagatcaacttctgtcatctagcaaatacaccatccaacggagcacaggagatagtattgatactcctaattatgatgtgcagaaacacatcaataagttatgtggcatgttattaatcacagaagatgctaatcataaattcactgggttaataggtatgttatatgctatgtctaggttaggaagagaagacaccataaaaatactcagagatgcgggatatcatgtaaaagcaaatggagtagatgtaacaacacatcgtcaagacatcaatgggaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgattctcctgattgtgggatgataatattatgtatagcagcattagtaataaccaaattggcagcaggggatagatctggtcttacagccgtgattaggagagctaataatgtcctaaaaaatgaaatgaaacgttacaaaggcttactacccaaggatatagccaacagcttctatgaagtgtttgaaaaacatccccactttatagatgtttttgttcattttggtatagcacaatcttccaccagaggtggcagtagagttgaagggatttttgcaggattgtttatgaatgcctatggtgcagggcaagtaatgctacggtggggagtcttagcaaaatcagttaaaaatattatgttaggacatgctagtgtgcaagcagaaatggaacaagttgttgaggtttatgaatatgcccaaaaattgggtggagaagcaggattctaccatatattgaacaacccaaaagcatcattattatctttgactcaatttcctcacttttccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtattagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacctaaagatcccaagaaaaaagatagtatcatatctgtcaactcaatagatatagaagtaaccaaagaaagccctataacatcaaattcaaccattattaacccaacaaatgagacagatgataatgcagggaacaagcccaattatcaaagaaaacctctagtaagtttcaaagaagaccctataccaagtgataatcccttttcaaaactatacaaagaaaccatagagacatttgataacaatgaagaagaatctagctattcatatgaagaaataaatgatcagacgaacgataatataactgcaagattagataggattgatgaaaaattaagtgaaatactaggaatgcttcacacattagtagtagcaagtgcaggacctacatctgctagggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctgttggaagggaatgatagtgacaatgatctatcacttgaagatttctgattagttacaaatctgcacttcaacacacaacaccaacagaagaccaacaaacaaaccaacccactcatccaaccaaacatccatccgccaatcagccaaacagccaacaaaacaaccagccaatccaaaaccagccacctggaaaaaatcgacaatatagttacaaaaaaagaaaagggtggggcaaatatggaaacatacgtgaacaagcttcacgaaggctccacatacacagctgctgttcaatacaatgtcctagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcattgctagcacaaatgcccagcaaatttaccatatgtgctaatgtgtccttggatgaaagaagcaaactggcatatgatgtaaccacaccctgtgaaatcaaggcatgtagtctaacatgcctaaaatcaaaaaatatgttaactacagttaaagatctcactatgaagacactcaaccccacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactattagtcatcacagtgactgacaacaaaggagcattcaaatacataaagccgcaaagtcaattcatagtagatcttggagcttacctagaaaaagaaagtatatattatgttaccacaaattggaagcacacagctacacgatttgcaatcaaacccatggaagattaacctttttcctccacatcagtgagtcaattcatacaaactttctacctacattcttcacttcaccattacaatcacaaacactctgtggttcaaccaatcaaacaaaacttatctgaagtctcagatcatcccaagtcattgttcatcagatctagtaatcaaataagttaataaaaatatacacatggggcaaataatcatcggaggaaatccaactaatcacaatatctgttaacatagacaagtcaacacaccagacagaatcaaccaatggaaaatacatccataacaatagaattctcaagcaaattctggccttactttacactaatacacatgatcacaacaataatctctttgctaatcataatctccatcatgactgcaatactaaacaaactttgtgaatataacgtattccataacaaaacctttgagttaccaagagctcgagtcaacacatagcattcatcaatctaatagctcaaaatagtaaccttgcatttaaaagtgaacaacccccacctctttacaacacctcattaacatcccaccatgcaaaccaccatccatactataaagtagttaattaaaaatagtcataacaatgaactaggatatcaagactaacaataacgttggggcaaatgcaaacatgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtagttattaaaaaacatattatcacaaaaggccatgaccaactcaaacagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaccaagaattagataaatataaaaatgctgtaacagaattgcagttgctcatgcaaagcacaacagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacatcatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaataccagattaacttactatctgtaaaaatgagaactggggcaaatatgtcacgaaggaatccttgcaaatttgaaattcgaggtcattgcttgaatggtaagagatgtcattttagtcataattattttgaatggccaccccatgcactgctcgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaataatataactaaacaatcagcatgtgttgccatgagcaaactcctcactgaactcaatagtgatgatatcaaaaaactgagagacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccacaagagcataaccatcaacaacccaaaagaattaactgttagtgatacaaatgaccatgccaaaaataatgatactacctgacaaatatccttgtagtataacttccatactaataacaagtagatgtagagtcactatgtataatcgaaagaacacactatatttcaatcaaaacaacccaaataaccatatgtactcaccgaatcaaacattcaatgaaatccattggacctcacaagacttgattgacacaattcaaaattttctacagcatctaggtgttattgaggatatatatacaatatatatattagtgtcataacactcaatcctaatactgaccatatcgttgaattattaattcaaataattcaagctgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagagcctacttattttcagtcattacttatgacatacaagagtatgacctcgttggaacagattgctaccactaatttacttaaaaagataataagaagagctatagaaataagtgatgtcaaagtctatgctatattgaataaactagggcttaaagaaaaggacaagattaaatccaacaatggacaggatgaagacaactcagttattacgaccataatcaaagatgatatactttcagctgttaaggataatcaatctcatcttaaagcagacaaaaatcactctacaaaacaaaaagacacaatcaaaacaacactcttgaagaaattaatgtgttcaatgcagcatcctccatcatggttaatacattggtttaatttatacacaaaattaaacaacatattaacacagtatcgatcaaatgaggttaaaaaccatgggtttatattgatagataatcaaactcttagtggatttcaatttattttgaatcaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggtgattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtatccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaagttaattgcaatgagaccaaattttacttgttaagcagtttgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcaccccctaaaaatttgatatggactagtttccctagaaattatatgccgtcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataagagatcatattgtagatcttaacaatgtagatgaacaaagtggattatatagatatcacatgggtggtattgaagggtggtgtcaaaaactatggaccatagaagctatatcactattggatctaatatctctcaaagggaaattctcaattactgctttaattaatggtgacaatcaatcaatagatataagcaaaccagtcagactcatggaaggtcaaactcatgctcaagcagattatttgctagcattaaatagccttaaattactgtataaagagtatgcaggcataggtcacaaattaaaaggaactgagacttatatatcacgagatatgcaatttatgagtaaaacaattcaacataacggtgtatattaccctgctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcaaattgctctacaattaaaaaatcatgcgttatgtaacaataaattatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgatttcctcacagaggctatagttcactctgtgttcatacttagttattatacaaaccatgacttaaaagataaacttcaagatttgtcagatgatagattgaataagttcttaacatgcataatcacgtttgacaaaaaccctaatgctgaattcgtaacattgatgagagatcctcaagctttagggtctgagagacaagctaaaattactagtgaaatcaatagactggcagttacagaggttttgagtacagctccaaacaaaatattctccaaaagtgcacaacattataccactacagagatagatctaaatgatattatgcaaaatatagaacctacatatcctcacgggctaagagttgtttatgaaagtttacccttttataaagcagagaaaatagtaaatcttatatcaggtacaaaatctataactaacatactggaaaagacttctgccatagacttaacagatattgatagagccactgagatgatgaggaaaaacataactttgcttataaggatacttccattggattgtaacagagataaaagagaaatattgagtatggaaaacctaagtattactgaattaagcaaatatgttagggaaagatcttggtctttatccaatatagttggtgttacatcacccagtatcatgtatacaatggacatcaaatatacaacaagcactatagctagtggcataattatagagaaatataatgttaacagtttaacacgtggtgagagaggaccaactaaaccatgggttggttcatctacacaagagaaaaaaacaatgccagtttataatagacaagttttaaccaaaaaacaaagagatcaaatagatctattagcaaaattggattgggtgtatgcatctatagataacaaggatgaattcatggaagaactcagcataggaacccttgggttaacatatgaaaaggccaaaaaattatttccacaatatttaagtgtcaactatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagcttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaacaaaacactcaaatctggatctcatgttaattctaatttaatattggcacataaaatatctgactattttcataatacttacattttaagtactaatttagctggacattggattctaattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaaactggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaatttacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttttacattaattataacttctcagataatactcatctattaactaaacatataaggattgctaattcagaattagaaaataattacaacaaattatatcatcctacaccagaaaccctagagaatatactagccaatccgattaaaagtaatgacaaaaagacactgaacgactattgtataggtaaaaatgttgactcaataatgttaccattgttatctaataagaagcttgttaaatcgtctgcaatgattagaaccaattacagcaaacaagacctgtacaatctattccctacggttgtgatcgatagaattatagatcattcaggtaatacagccaaatccaaccaactttacactactacttcccatcaaatatctttagtgcacaatagcacatcactttattgcatgcttccttggcatcatattaatagattcaattttgtatttagttctacaggttgtaaaattagtatagagtatattttaaaagaccttaaaattaaagatcctaattgtatagcattcataggtgaaggagcagggaatttattattgcgtacagtggtggaacttcatcctgacataagatatatttacagaagtctgaaagattgcaatgatcatagtttacctattgagtttttaaggctatacaatggacatatcaacattgattatggtgaaaatttgaccattcctgctacagatgcaaccaacaacattcattggtcttatttacatataaagtttgctgaacctatcagtctttttgtatgtgatgccgaattgcctgtaacagtcaactggagtaaaattataatagaatggagcaagcatgtaagaaaatgcaagtactgttcctcagttaataaatgtacgttaatagtaaaatatcatgctcaagatgatattgatttcaaattagacaatataactatattaaaaacttatgtatgcttaggcagtaagttaaagggatcggaggtttacttagtccttacaataggtcctgcaaatatatttccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacggaatgaagttttcagcaataaacttataaatcataagcatatgaacatcttaaagtggttcaatcatgttttaaatttcagatcaacagaactaaactataaccatttatatatggtagaatctacatatccttacctaagtgaattgttaaacagcttgacaactaatgaacttaaaaaactgattaaaatcacaggtagtctgttatacaactttcataatgaataatgaataaagatcttataataaaaattcctatagctatacactagcactgtattcaattatagttattaaaaaattaaaaatcatataattttttataaaaataacttttagtgaactaatcctaaagttatcattttgatctaggaggaataaatttaaatcccaatctaattggtttatatgtgtattaactaaactacgagatattagtttttgacactttttttctcgt

SEQ ID NO: 7 - DNA, 18 bp, Artificial Sequence
  Synthetic Oligonucleotide
atgcataaggtgactcct

SEQ ID NO: 8 - DNA, 18 bp, Artificial Sequence
  Synthetic Oligonucleotide
ttactgccgtggggtgtt

SEQ ID NO: 9 - DNA, 19 bp, Artificial Sequence
  Synthetic Oligonucleotide
acaggagaagggacgccat

SEQ ID NO: 10 - DNA, 21 bp, Artificial Sequence
  Synthetic Oligonucleotide
gaagccctacagacgagctca

SEQ ID NO: 11 - DNA, 23 bp, Artificial Sequence
  Synthetic Oligonucleotide
agcacagtggtgaaagagacctt

SEQ ID NO: 12 - DNA, 22 bp, Artificial Sequence
  Synthetic Oligonucleotide
tccaatgcatagctggtgattt

SEQ ID NO: 13 - DNA, 21 bp, Artificial Sequence
  Synthetic Oligonucleotide
ggagctgagcaacatcacaca

SEQ ID NO: 14 - DNA, 21 bp, Artificial Sequence
  Synthetic Oligonucleotide
ggtcctgtagatggcattgca

SEQ ID NO: 15 - DNA, 24 bp, Artificial Sequence
  Synthetic Oligonucleotide
tcaagtggcatagatgtggaagaa

SEQ ID NO: 16 - DNA, 21 bp, Artificial Sequence
  Synthetic Oligonucleotide
tggctctgcaggattttcatg

SEQ ID NO: 17 - DNA, 20 bp, Artificial Sequence
  Synthetic Oligonucleotide
ctggtgaaaaggacctctcg

SEQ ID NO: 18 - DNA, 26 bp, Artificial Sequence
  Synthetic Oligonucleotide
tgaagtactcattatagtcaagggca

SEQ ID NO: 19 - DNA, 897 bp, Human respiratory syncytial virus
atgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtag

SEQ ID NO: 20 - DNA, 702 bp, Artificial Sequence
  Synthetic Oligonucleotide
atgcacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtag

SEQ ID NO: 21 - DNA, 702 bp, Artificial Sequence
  Synthetic Oligonucleotide
atgcataaagtaaccccgaccaccgctatcatccaggacgctaccagccagatcaaaaacactacgcctacctatctgactcagaacccgcaactgggcatctccccgtccaatccgtctgaaattacctcccagatcactaccatcctggcatccactactccgggtgtgaaatctaccctgcagtccactaccgtaaaaacgaaaaacaccaccactacccagactcagccttccaaacctactacgaaacagcgtcagaacaaaccgccgagcaaaccgaacaacgacttccactttgaagttttcaacttcgtcccatgcagcatttgtagcaacaatccgacctgctgggcaatttgcaaacgcatcccaaacaaaaagccgggcaaaaagacgaccactaaaccaaccaagaaacctaccctgaaaactaccaaaaaagacccgaaaccgcagaccaccaaatctaaagaagttccgacgaccaaaccgaccgaggaaccgacgatcaacaccacgaaaacgaacatcatcaccaccctgctgacctctaacactaccggtaatccggagctgactagccagatggaaacctttcacagcacttcttctgaaggtaacccatctccgagccaggtgtccaccacttctgaatacccgagccaaccgtcctccccgcctaatacgccgcgtcaataa

SEQ ID NO: 22 - DNA, 702 bp, Artificial Sequence
  Synthetic Oligonucleotide
atgcataaggtgactcctacaacggctatcattcaggacgccacctcccaaatcaaaaacactacacccacttatctgacacagaacccccaactgggcatcagcccttccaacccttctgaaatcacttcccagatcaccactatcttggcttctactacccctggggtcaagtccactctgcagtctaccacagtcaaaacaaagaatacaaccactacccagactcagccaagcaagccaacaacaaagcagcgacaaaataaaccccctagtaagccaaataacgacttccactttgaggtgtttaattttgttccttgcagtatctgctctaacaatcccacctgttgggcgatatgtaaacgcatcccgaataagaagccaggtaagaagacaaccacaaagcccacaaagaaacccaccctgaaaacaaccaagaaagatccaaagccccagacgaccaaaagcaaagaggtgcctacgacaaagccgacagaagagcctacaatcaataccaccaagaccaacattattaccacccttcttacttctaacactaccggaaatcctgagttgacaagtcagatggagacattccattcaacgtcctcagaaggcaacccaagcccctcccaggtatcaaccacctctgaatacccgagccagccctccagtcccccaaacaccccacggcagtaa

SEQ ID NO: 23 - DNA, 699 bp, Artificial Sequence
  Synthetic Oligonucleotide
atggccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatggaggaggaggaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaattaa

SEQ ID NO: 24 - DNA, 699 bp, Artificial Sequence
  Synthetic Oligonucleotide
atggctattgtaagctgctatggtaagactaaatgcactgcgagcaataaaaaccgtggtattatcaaaacctttagcaacggctgtgattacgtatccaacaaaggcgttgacactgtttctgtgggcaacaccctgtattacgtgaacaagcaggaaggcaaaagcctgtacgtgaaaggtgaaccgattatcaacttttacgacccgctggtcttcccgtctgatgagttcgatgcttctatcagccaggttaacgaaaagatcaatcagtctctggctttcatccgtaaaagcgatgagctgctgcataacgtcaacgctggtaaatctaccactaacggtggtggcggtggcgctattgttagctgctacggtaaaacgaaatgcaccgctagcaacaaaaatcgtggcatcatcaaaacgttctctaacggttgcgactatgtttctaacaaaggtgtagacactgtgtctgtgggtaacactctgtactacgttaacaaacaggaaggtaagtctctgtacgttaaaggcgagccgatcatcaacttctacgacccactggtttttccatctgacgaatttgacgcatctattagccaggtgaacgagaaaatcaaccagagcctggcgttcatccgcaaatccgacgaactgctgcacaacgttaacgctggcaaatccaccacgaactaa

SEQ ID NO: 25 - protein, 232 aa, Artificial Sequence
  Synthetic Peptide
MAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGGGGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 26 - DNA, 1725 bp, Human respiratory syncytial virus
atggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaccaagaattagataaatataaaaatgctgtaacagaattgcagttgctcatgcaaagcacaacagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacatcatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactga

SEQ ID NO: 27 - DNA, 420 bp, Human respiratory syncytial virus
atgggcagcaattcgttgagtatgataaaagttagattacaaaatttgtttgacaatgatgaagtagcattgttaaaaataacatgctatactgacaaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtgctacaaaatggaggttatatatgggaaatgatggaattaacacattgctctcaacctaatggtctaatagatgacaattgtgaaattaaattctccaaaaaactaagtgattcaacaatgaccaattatatgaatcaattatctgaattacttggatttgatcttaatccataa

SEQ ID NO: 28 - protein, 139 aa, Human respiratory syncytial virus
MGSNSLSMIKVRLQNLFDNDEVALLKITCYTDKLIHLTNALAKAVIHTIKLNGIVFVHVITSSDICPNNNIVVKSNFTTMPVLQNGGYIWEMMELTHCSQPNGLIDDNCEIKFSKKLSDSTMTNYMNQLSELLGFDLNP

SEQ ID NO: 29 - DNA, 375 bp, Human respiratory syncytial virus
atggacacaacccacaatgataccacaccacaaagactgatgatcacagacatgagaccgttgtcacttgagactacaataacatcactaaccagagacatcataacacacagatttatatacttaataaatcatgaatgcatagtgagaaaacttgatgaaagacaggccacatttacattcctggtcaactatgaaatgaaactattgcacaaagtaggaagcactaaatataaaaaatatactgaatacaacacaaaatatggcactttccctatgccgatattcatcaatcatgatgggttcttagaatgcattggcattaagcctacaaagcatactcccataatatacaagtatgatctcaatccatga

SEQ ID NO: 30 - protein, 124 aa, Human respiratory syncytial virus
MDTTHNDTTPQRLMITDMRPLSLETTITSLTRDIITHRFIYLINHECIVRKLDERQATFTFLVNYEMKLLHKVGSTKYKKYTEYNTKYGTFPMPIFINHDGFLECIGIKPTKHTPIIYKYDLNP

SEQ ID NO: 31 - DNA, 1176 bp, Human respiratory syncytial virus
atggctcttagcaaagtcaagttgaatgatacactcaacaaagatcaacttctgtcatctagcaaatacaccatccaacggagcacaggagatagtattgatactcctaattatgatgtgcagaaacacatcaataagttatgtggcatgttattaatcacagaagatgctaatcataaattcactgggttaataggtatgttatatgctatgtctaggttaggaagagaagacaccataaaaatactcagagatgcgggatatcatgtaaaagcaaatggagtagatgtaacaacacatcgtcaagacatcaatgggaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgattctcctgattgtgggatgataatattatgtatagcagcattagtaataaccaaattggcagcaggggatagatctggtcttacagccgtgattaggagagctaataatgtcctaaaaaatgaaatgaaacgttacaaaggcttactacccaaggatatagccaacagcttctatgaagtgtttgaaaaacatccccactttatagatgtttttgttcattttggtatagcacaatcttccaccagaggtggcagtagagttgaagggatttttgcaggattgtttatgaatgcctatggtgcagggcaagtaatgctacggtggggagtcttagcaaaatcagttaaaaatattatgttaggacatgctagtgtgcaagcagaaatggaacaagttgttgaggtttatgaatatgcccaaaaattgggtggagaagcaggattctaccatatattgaacaacccaaaagcatcattattatctttgactcaatttcctcacttttccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtattagacttgacagcagaagaactagaggctatcaaacatcagcttaatccaaaagataatgatgtagagctttga

SEQ ID NO: 32 - protein, 391 aa, Human respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 33 - DNA, 726 bp, Human respiratory syncytial virus
atggaaaagtttgctcctgaattccatggagaagatgcaaacaacagggctactaaattcctagaatcaataaagggcaaattcacatcacctaaagatcccaagaaaaaagatagtatcatatctgtcaactcaatagatatagaagtaaccaaagaaagccctataacatcaaattcaaccattattaacccaacaaatgagacagatgataatgcagggaacaagcccaattatcaaagaaaacctctagtaagtttcaaagaagaccctataccaagtgataatcccttttcaaaactatacaaagaaaccatagagacatttgataacaatgaagaagaatctagctattcatatgaagaaataaatgatcagacgaacgataatataactgcaagattagataggattgatgaaaaattaagtgaaatactaggaatgcttcacacattagtagtagcaagtgcaggacctacatctgctagggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctgttggaagggaatgatagtgacaatgatctatcacttgaagatttctga

SEQ ID NO: 34 - protein, 241 aa, Human respiratory syncytial virus
MEKFAPEFHGEDANNRATKFLESIKGKFTSPKDPKKKDSIISVNSIDIEVTKESPITSNSTIINPTNETDDNAGNKPNYQRKPLVSFKEDPIPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDF

SEQ ID NO: 35 - DNA, 771 bp, Human respiratory syncytial virus
atggaaacatacgtgaacaagcttcacgaaggctccacatacacagctgctgttcaatacaatgtcctagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcattgctagcacaaatgcccagcaaatttaccatatgtgctaatgtgtccttggatgaaagaagcaaactggcatatgatgtaaccacaccctgtgaaatcaaggcatgtagtctaacatgcctaaaatcaaaaaatatgttaactacagttaaagatctcactatgaagacactcaaccccacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactattagtcatcacagtgactgacaacaaaggagcattcaaatacataaagccgcaaagtcaattcatagtagatcttggagcttacctagaaaaagaaagtatatattatgttaccacaaattggaagcacacagctacacgatttgcaatcaaacccatggaagattaa

SEQ ID NO: 36 - protein, 256 aa, Human respiratory syncytial virus
METYVNKLHEGSTYTAAVQYNVLEKDDDPASLTIWVPMFQSSMPADLLIKELANVNILVKQISTPKGPSLRVMINSRSALLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 37 - DNA, 195 bp, Human respiratory syncytial virus
atggaaaatacatccataacaatagaattctcaagcaaattctggccttactttacactaatacacatgatcacaacaataatctctttgctaatcataatctccatcatgactgcaatactaaacaaactttgtgaatataacgtattccataacaaaacctttgagttaccaagagctcgagtcaacacatag

SEQ ID NO: 38 - protein, 64 aa, Human respiratory syncytial virus
MENTSITIEFSSKFWPYFTLIHMITTIISLLIIISIMTAILNKLCEYNVFHNKTFELPRARVNT

SEQ ID NO: 39 - DNA, 585 bp, Human respiratory syncytial virus
atgtcacgaaggaatccttgcaaatttgaaattcgaggtcattgcttgaatggtaagagatgtcattttagtcataattattttgaatggccaccccatgcactgctcgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaataatataactaaacaatcagcatgtgttgccatgagcaaactcctcactgaactcaatagtgatgatatcaaaaaactgagagacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccacaagagcataaccatcaacaacccaaaagaattaactgttagtgatacaaatgaccatgccaaaaataatgatactacctga

SEQ ID NO: 40 - protein, 194 aa, Human respiratory syncytial virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKELTVSDTNDHAKNNDTT

SEQ ID NO: 41 - DNA, 273 bp, Human respiratory syncytial virus
atgaccatgccaaaaataatgatactacctgacaaatatccttgtagtataacttccatactaataacaagtagatgtagagtcactatgtataatcgaaagaacacactatatttcaatcaaaacaacccaaataaccatatgtactcaccgaatcaaacattcaatgaaatccattggacctcacaagacttgattgacacaattcaaaattttctacagcatctaggtgttattgaggatatatatacaatatatatattagtgtcataa

SEQ ID NO: 42 - protein, 90 aa, Human respiratory syncytial virus
MTMPKIMILPDKYPCSITSILITSRCRVTMYNRKNTLYFNQNNPNNHMYSPNQTFNEIHWTSQDLIDTIQNFLQHLGVIEDIYTIYILVS

SEQ ID NO: 43 - DNA, 6498 bp, Human respiratory syncytial virus
atggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagagcctacttattttcagtcattacttatgacatacaagagtatgacctcgttggaacagattgctaccactaatttacttaaaaagataataagaagagctatagaaataagtgatgtcaaagtctatgctatattgaataaactagggcttaaagaaaaggacaagattaaatccaacaatggacaggatgaagacaactcagttattacgaccataatcaaagatgatatactttcagctgttaaggataatcaatctcatcttaaagcagacaaaaatcactctacaaaacaaaaagacacaatcaaaacaacactcttgaagaaattaatgtgttcaatgcagcatcctccatcatggttaatacattggtttaatttatacacaaaattaaacaacatattaacacagtatcgatcaaatgaggttaaaaaccatgggtttatattgatagataatcaaactcttagtggatttcaatttattttgaatcaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggtgattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtatccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaagttaattgcaatgagaccaaattttacttgttaagcagtttgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcaccccctaaaaatttgatatggactagtttccctagaaattatatgccgtcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataagagatcatattgtagatcttaacaatgtagatgaacaaagtggattatatagatatcacatgggtggtattgaagggtggtgtcaaaaactatggaccatagaagctatatcactattggatctaatatctctcaaagggaaattctcaattactgctttaattaatggtgacaatcaatcaatagatataagcaaaccagtcagactcatggaaggtcaaactcatgctcaagcagattatttgctagcattaaatagccttaaattactgtataaagagtatgcaggcataggtcacaaattaaaaggaactgagacttatatatcacgagatatgcaatttatgagtaaaacaattcaacataacggtgtatattaccctgctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcaaattgctctacaattaaaaaatcatgcgttatgtaacaataaattatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgatttcctcacagaggctatagttcactctgtgttcatacttagttattatacaaaccatgacttaaaagataaacttcaagatttgtcagatgatagattgaataagttcttaacatgcataatcacgtttgacaaaaaccctaatgctgaattcgtaacattgatgagagatcctcaagctttagggtctgagagacaagctaaaattactagtgaaatcaatagactggcagttacagaggttttgagtacagctccaaacaaaatattctccaaaagtgcacaacattataccactacagagatagatctaaatgatattatgcaaaatatagaacctacatatcctcacgggctaagagttgtttatgaaagtttacccttttataaagcagagaaaatagtaaatcttatatcaggtacaaaatctataactaacatactggaaaagacttctgccatagacttaacagatattgatagagccactgagatgatgaggaaaaacataactttgcttataaggatacttccattggattgtaacagagataaaagagaaatattgagtatggaaaacctaagtattactgaattaagcaaatatgttagggaaagatcttggtctttatccaatatagttggtgttacatcacccagtatcatgtatacaatggacatcaaatatacaacaagcactatagctagtggcataattatagagaaatataatgttaacagtttaacacgtggtgagagaggaccaactaaaccatgggttggttcatctacacaagagaaaaaaacaatgccagtttataatagacaagttttaaccaaaaaacaaagagatcaaatagatctattagcaaaattggattgggtgtatgcatctatagataacaaggatgaattcatggaagaactcagcataggaacccttgggttaacatatgaaaaggccaaaaaattatttccacaatatttaagtgtcaactatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagcttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaacaaaacactcaaatctggatctcatgttaattctaatttaatattggcacataaaatatctgactattttcataatacttacattttaagtactaatttagctggacattggattctaattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaaactggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaatttacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttttacattaattataacttctcagataatactcatctattaactaaacatataaggattgctaattcagaattagaaaataattacaacaaattatatcatcctacaccagaaaccctagagaatatactagccaatccgattaaaagtaatgacaaaaagacactgaacgactattgtataggtaaaaatgttgactcaataatgttaccattgttatctaataagaagcttgttaaatcgtctgcaatgattagaaccaattacagcaaacaagacctgtacaatctattccctacggttgtgatcgatagaattatagatcattcaggtaatacagccaaatccaaccaactttacactactacttcccatcaaatatctttagtgcacaatagcacatcactttattgcatgcttccttggcatcatattaatagattcaattttgtatttagttctacaggttgtaaaattagtatagagtatattttaaaagaccttaaaattaaagatcctaattgtatagcattcataggtgaaggagcagggaatttattattgcgtacagtggtggaacttcatcctgacataagatatatttacagaagtctgaaagattgcaatgatcatagtttacctattgagtttttaaggctatacaatggacatatcaacattgattatggtgaaaatttgaccattcctgctacagatgcaaccaacaacattcattggtcttatttacatataaagtttgctgaacctatcagtctttttgtatgtgatgccgaattgcctgtaacagtcaactggagtaaaattataatagaatggagcaagcatgtaagaaaatgcaagtactgttcctcagttaataaatgtacgttaatagtaaaatatcatgctcaagatgatattgatttcaaattagacaatataactatattaaaaacttatgtatgcttaggcagtaagttaaagggatcggaggtttacttagtccttacaataggtcctgcaaatatatttccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacggaatgaagttttcagcaataaacttataaatcataagcatatgaacatcttaaagtggttcaatcatgttttaaatttcagatcaacagaactaaactataaccatttatatatggtagaatctacatatccttacctaagtgaattgttaaacagcttgacaactaatgaacttaaaaaactgattaaaatcacaggtagtctgttatacaactttcataatgaataa

SEQ ID NO: 44 - protein, 2165 aa, Human respiratory syncytial virus
MDPIINGNSANVYLTDSYLKGVISFSECNALGSYIFNGPYLKNDYTNLISRQNPLIEHMNLKKLNITQSLISKYHKGEIKLEEPTYFQSLLMTYKSMTSLEQIATTNLLKKIIRRAIEISDVKVYAILNKLGLKEKDKIKSNNGQDEDNSVITTIIKDDILSAVKDNQSHLKADKNHSTKQKDTIKTTLLKKLMCSMQHPPSWLIHWFNLYTKLNNILTQYRSNEVKNHGFILIDNQTLSGFQFILNQYGCIVYHKELKRITVTTYNQFLTWKDISLSRLNVCLITWISNCLNTLNKSLGLRCGFNNVILTQLFLYGDCILKLFHNEGFYIIKEVEGFIMSLILNITEEDQFRKRFYNSMLNNITDAANKAQKNLLSRVCHTLLDKTVSDNIINGRWIILLSKFLKLIKLAGDNNLNNLSELYFLFRIFGHPMVDERQAMDAVKVNCNETKFYLLSSLSMLRGAFIYRIIKGFVNNYNRWPTLRNAIVLPLRWLTYYKLNTYPSLLELTERDLIVLSGLRFYREFRLPKKVDLEMIINDKAISPPKNLIWTSFPRNYMPSHIQNYIEHEKLKFSESDKSRRVLEYYLRDNKFNECDLYNCVVNQSYLNNPNHVVSLTGKERELSVGRMFAMQPGMFRQVQILAEKMIAENILQFFPESLTRYGDLELQKILELKAGISNKSNRYNDNYNNYISKCSIITDLSKFNQAFRYETSCICSDVLDELHGVQSLFSWLHLTIPHVTIICTYRHAPPYIRDHIVDLNNVDEQSGLYRYHMGGIEGWCQKLWTIEAISLLDLISLKGKFSITALINGDNQSIDISKPVRLMEGQTHAQADYLLALNSLKLLYKEYAGIGHKLKGTETYISRDMQFMSKTIQHNGVYYPASIKKVLRVGPWINTILDDFKVSLESIGSLTQELEYRGESLLCSLIFRNVWLYNQIALQLKNHALCNNKLYLDILKVLKHLKTFFNLDNIDTALTLYMNLPMLFGGGDPNLLYRSFYRRTPDFLTEAIVHSVFILSYYTNHDLKDKLQDLSDDRLNKFLTCIITFDKNPNAEFVTLMRDPQALGSERQAKITSEINRLAVTEVLSTAPNKIFSKSAQHYTTTEIDLNDIMQNIEPTYPHGLRVVYESLPFYKAEKIVNLISGTKSITNILEKTSAIDLTDIDRATEMMRKNITLLIRILPLDCNRDKREILSMENLSITELSKYVRERSWSLSNIVGVTSPSIMYTMDIKYTTSTIASGIIIEKYNVNSLTRGERGPTKPWVGSSTQEKKTMPVYNRQVLTKKQRDQIDLLAKLDWVYASIDNKDEFMEELSIGTLGLTYEKAKKLFPQYLSVNYLHRLTVSSRPCEFPASIPAYRTTNYHFDTSPINRILTEKYGDEDIDIVFQNCISFGLSLMSVVEQFTNVCPNRIILIPKLNEIHLMKPPIFTGDVDIHKLKQVIQKQHMFLPDKISLTQYVELFLSNKTLKSGSHVNSNLILAHKISDYFHNTYILSTNLAGHWILIIQLMKDSKGIFEKDWGEGYITDHMFINLKVFFNAYKTYLLCFHKGYGKAKLECDMNTSDLLCVLELIDSSYWKSMSKVFLEQKVIKYILSQDASLHRVKGCHSFKLWFLKRLNVAEFTVCPWVVNIDYHPTHMKAILTYIDLVRMGLINIDRIHIKNKHKFNDEFYTSNLFYINYNFSDNTHLLTKHIRIANSELENNYNKLYHPTPETLENILANPIKSNDKKTLNDYCIGKNVDSIMLPLLSNKKLVKSSAMIRTNYSKQDLYNLFPTVVIDRIIDHSGNTAKSNQLYTTTSHQISLVHNSTSLYCMLPWHHINRFNFVFSSTGCKISIEYILKDLKIKDPNCIAFIGEGAGNLLLRTVVELHPDIRYIYRSLKDCNDHSLPIEFLRLYNGHINIDYGENLTIPATDATNNIHWSYLHIKFAEPISLFVCDAELPVTVNWSKIIIEWSKHVRKCKYCSSVNKCTLIVKYHAQDDIDFKLDNITILKTYVCLGSKLKGSEVYLVLTIGPANIFPVFNVVQNAKLILSRTKNFIMPKKADKESIDANIKSLIPFLCYPITKKGINTALSKLKSVVSGDILSYSIAGRNEVFSNKLINHKHMNILKWFNHVLNFRSTELNYNHLYMVESTYPYLSELLNSLTTNELKKLIKITGSLLYNFHNE

SEQ ID NO: 45 - DNA, 909 bp, Human respiratory syncytial virus
aatgcaaccatgtccaaacacaagaatcaacgcactgccaggactctagaaaagacctgggatactcttaatcatctaattgtaatatcctcttgtttatacagattaaatttaaaatctatagcacaaatagcactatcagtattggcaatgataatctcaacctctctcataattgcagccataatattcatcatctctgccaatcacaaagttacactaacaacggtcacagtttcaacaataaaaaaccacactgaaaaaaacatcaccacttaccttactcaagtctcaccagaaagagttagcccatccaaacaacccacaaccacatcaccaatccacacaaactcagccacaatatcacccaatacaaaatcagaaacacaccatacaacagcgcaaaccaaaggcagaatcaccactccaacacagaccaacaagccaagcacaaaaccacgtccaaaaattccaccaaaaaaagatgattaccattttgaagtgttcaacttcgttccctgtagtatatgtggcaacaatcgactttgcaaatccatctgcaaaacaataccaagcaacaaaccaaagaaaaaaccaaccatcaaacctacaaacaaaccaactaccaaaaccacaaacaaaatagacccaaaaacaccagccaaaacaccgaaaaaagaaactaccaccaacccaacaaaaaaaccaaccctcaagatcacagaaaaagacaccagcacttcacaatccactatgctcgacacaaccaaaccaaatcacacaatccaacagcaatacctccactcaaccacccccgataacacacccaactccacacaaacacccacagcatccgagccctccacatcaaactcaacccaagaagtctagtcacatgcttagttattc

SEQ ID NO: 46 - protein, 293 aa, Human respiratory syncytial virus
MSKHKNQRTARTLEKTWDTLNHLIVISSCLYRLNLKSIAQIALSVLAMIISTSLIIAAIIFIISANHKVTLTTVTVSTIKNHTEKNITTYLTQVSPERVSPSKQPTTTSPIHTNSATISPNTKSETHHTTAQTKGRITTPTQTNKPSTKPRPKIPPKKDDYHFEVFNFVPCSICGNNRLCKSICKTIPSNKPKKKPTIKPTNKPTTKTTNKIDPKTPAKTPKKETTTNPTKKPTLKITEKDTSTSQSTMLDTTKPNHTIQQQYLHSTTPDNTPNSTQTPTASEPSTSNSTQEV

SEQ ID NO: 47 - DNA, 975 bp, Human respiratory syncytial virus
aatgcaaccatgtccaaacacaagaatcaacgcactgccaggactctagaaaagacctgggatactcttaatcatctaattgtaatatcctcttgtttatacaaattaaatttaaaatctatagcacaaatagcactatcagttttggcaatgataatctcaacctctctcataattgcagccataatattcatcatctctgccaatcacaaagttacactaacaactgtcacagttcaaacaataaaaaaccacactgagaaaaacatcaccacttaccttactcaagtctcaccagaaagggttagcccatccaaacaacccacaaccacaccaccaatccacacaaactcagccacaatatcacctaatacaagatcagaaacacaccatacaacagcacaaaccaaaggcagaaccaccactccgacacagaacaacaagccaagcacaaaaccacgtccaaaaaatccaccaaaaaaaccaaaagatgattaccattttgaagtgttcaacttcgttccctgtagtatatgtggcaacaatcaactctgcaaatccatttgcaaaacaataccaagcaataaaccaaagaaaaaaccaaccataaaacccacaaacaaaccacccaccaaaaccacaaccaaaagagacccaaaaacactagccaaaacactgaaaaaagaaaccaccatcaacccaacaaaaaaaccaacccccaagaccacagaaagagacaccagtaccccacaatccactgtgctcgacacaaccacatcaaaacacacaggaagagacaccagcacctcacaatccattgtgctcgacacaaccacatcaaaacacacaatccaacagcaatccctctactcaaccacccccgaaaacacacccaactccacacaaacgcccacagcatccgagccctccacatcaaattccacccaaaaactctagtcacatgcttagttattc

SEQ ID NO: 48 - protein, 315 aa, Human respiratory syncytial virus
MSKHKNQRTARTLEKTWDTLNHLIVISSCLYKLNLKSIAQIALSVLAMIISTSLIIAAIIFIISANHKVTLTTVTVQTIKNHTEKNITTYLTQVSPERVSPSKQPTTTPPIHTNSATISPNTRSETHHTTAQTKGRTTTPTQNNKPSTKPRPKNPPKKPKDDYHFEVFNFVPCSICGNNQLCKSICKTIPSNKPKKKPTIKPTNKPPTKTTTKRDPKTLAKTLKKETTINPTKKPTPKTTERDTSTPQSTVLDTTTSKHTGRDTSTSQSIVLDTTTSKHTIQQQSLYSTTPENTPNSTQTPTASEPSTSNSTQKL

SEQ ID NO: 49 - DNA, 909 bp, Human respiratory syncytial virus
aatgcaaccatgtccaaacacaagaatcaacgcactgccaggactctagaaaagacctgggatactcttaatcatctaattgtaatatcctcttgtttatacaggttaaatttaaaatctatagcacaaatagcactatcagtattggcaatgataatctcaacctctctcataattgcagccataatattcatcatctctgccaatcacaaagttacactaacaacggtcacagtttcaacaataaaaagccacactgaaaaaaacatcaccacttaccttactcaagtctcaccagaaagggttagcccatccaaacaacccacaaccacatcaccaatccacacaaactcagccacaatatcacccaatacaaaatcagaaacacaccatacaacagcacaaaccaaaggcagattcaccactccaacacagaccaacaagccaagcacaaaaccacgtccaaaaattccaccaaaaaaagatgattaccattttgaagtgttcaacttcgttccctgtagtatatgtggcaacaatcgactttgcaaatccatctgcaaaacaataccaagcaacaaaccaaagaaaaaaccaaccatcaaacctacaaacaaaccaaccaccaaaaccacaaacaaaatagacccaaaaacaccagccaaaacaccggaaaaagaaactaccaccaactcaacaaaaaaaccaaccctcaagatcacagaaaaagacaccagcacctcacaatccactatgctcgacacaaccacaccaaatcacacaatccaacagcaatccctccactcaaccacccccgataacacacccaactccacacaaacacccacagcatccgagccctccacatcaaactcaacccaaaaagtctagtcacatgcttagttattc

SEQ ID NO: 50 - protein, 293 aa, Human respiratory syncytial virus
MSKHKNQRTARTLEKTWDTLNHLIVISSCLYRLNLKSIAQIALSVLAMIISTSLIIAAIIFIISANHKVTLTTVTVSTIKSHTEKNITTYLTQVSPERVSPSKQPTTTSPIHTNSATISPNTKSETHHTTAQTKGRFTTPTQTNKPSTKPRPKIPPKKDDYHFEVFNFVPCSICGNNRLCKSICKTIPSNKPKKKPTIKPTNKPTTKTTNKIDPKTPAKTPEKETTTNSTKKPTLKITEKDTSTSQSTMLDTTTPNHTIQQQSLHSTTPDNTPNSTQTPTASEPSTSNSTQKV

SEQ ID NO: 51 - DNA, 915 bp, Human respiratory syncytial virus
aatgcaaccatgtccaaacacaagaatcaacgcactgccaggactctagaaaagacctgggatactcttaatcatctaattgtaatatcctcttgtttatacaaattaaatttaaaatctatagcacaaatagcactatcagttttggcaatgataatctcaacctctctcataattgcagccataatattcatcatctctgccaatcacaaagttacactaacaacggtcacagttcaaacaataaaaaaccacactgaaaaaaacatcacctcttaccttactcaagtctcaccagaaagggccagcccatccaaacaacccacaaccacaccaccaatccacacaaactcagccacaacatcacccaacacaaaatcagaaacacaccatacaacagcacaaaccaaaggcagaaccaccactccaacacagaacaacaagccaagcacaaaaccacgtccaaaaaatccaccaaaaaaaccaaaagatgattaccattttgaagtgttcaacttcgttccctgtagtatatgtggcaacaatcaactttgcagatccatctgcaaaacaataccaagcaataaaccaaagaaaaaaccaaccatcaaacccacaaacaaaccacccaccaaaaccacaaacaaaagagacccaaaaacaccagcaaaaccactgaaaaaagaaaccaccaccaacccaacaaaaaaaccaacccccaagaccacagaaagagactccagcacttcacaatccactgtgctcgacacaaccacatcaaaacacacaatccaacagcaatccctccactcaaccacccccgaaaacacacccaactccacacaaacacccacagcatccgagccctccacatcaaattccacccaagaaccctagtcacatgcttagttattc

SEQ ID NO: 52 - protein, 295 aa, Human respiratory syncytial virus
MSKHKNQRTARTLEKTWDTLNHLIVISSCLYKLNLKSIAQIALSVLAMIISTSLIIAAIIFIISANHKVTLTTVTVQTIKNHTEKNITSYLTQVSPERASPSKQPTTTPPIHTNSATTSPNTKSETHHTTAQTKGRTTTPTQNNKPSTKPRPKNPPKKPKDDYHFEVFNFVPCSICGNNQLCRSICKTIPSNKPKKKPTIKPTNKPPTKTTNKRDPKTPAKPLKKETTTNPTKKPTPKTTERDSSTSQSTVLDTTTSKHTIQQQSLHSTTPENTPNSTQTPTASEPSTSNSTQEP

SEQ ID NO: 53 - DNA, 1725 bp, Human respiratory syncytial virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 54 - protein, 574 aa, Human respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN